(19) United States
(12) Patent Application Publication
(10) Pub. No.: US 2025/0120986 A1
(43) Pub. Date: Apr. 17, 2025

(54) STEROL THERAPY

(71) Applicants: UCL BUSINESS LTD, London (GB); JENA UNIVERSITY HOSPITAL, Jena (DE)

(72) Inventors: Mervyn Singer, London (GB); Anna Kleyman, London (GB); Adrian Press, Jena (DE); Michael Bauer, Jena (DE)

(21) Appl. No.: 18/688,602

(22) PCT Filed: Sep. 13, 2022
(86) PCT No.: PCT/GB2022/052309

§ 371 Date: Mar. 1, 2024

(30) Foreign Application Priority Data
Sep. 13, 2021 (GB) 2113028.1

(51) Int. Cl. A61K 31/575; A61K 9/127

(52) U.S. Cl. CPC A61K 31/575; A61K 9/127

(57) ABSTRACT

The present invention relates to methods of treating myocardial depression or increasing adrenergic signalling responsiveness in a subject. The methods involve administering to a subject in need thereof a therapeutically effective amount of a composition comprising one or more compounds of formula (II), for example one or more compounds of formula (I), described herein. The present invention is also concerned with compositions comprising cholesterol and/or one or more phytosterols for use in methods of treating myocardial depression or increasing adrenergic signalling responsiveness in a subject.

### FIELD OF THE INVENTION

The present invention is concerned with methods of treating myocardial depression or increasing adrenergic signalling responsiveness in a subject. The methods involve administering to a subject in need thereof a therapeutically effective amount of a composition comprising one or more compounds of formula (II), for example one or more compounds of formula (I), described herein. The present invention is also concerned with compositions comprising cholesterol and/or one or more phytosterols for use in methods of treating myocardial depression or increasing adrenergic signalling responsiveness in a subject.

The present invention is also concerned with pharmaceutical compositions comprising a blend of cholesterol and one or more other compounds of formula (II), for example one or more compounds of formula (I).

### BACKGROUND TO THE INVENTION

### Cholesterol and Phytosterol

Cholesterol plays pleiotropic roles within the body including production of hormones, vitamin D and bile acids, anti-inflammatory/immunomodulatory activity. Crucially, it is an integral component of cell membranes, dictating membrane fluidity and thus the activity of multiple membrane receptors including many G-protein coupled receptors, including the beta-adrenergic receptor.

Phytosterols are cholesterol analogues derived from plants, with examples including sitosterol and stigmasterol. Phytosterols are known in the art as having a similar structure and similar functions to cholesterol. Cholesterol and phytosterols are compounds that have formula (II) described herein.

### Myocardial Depression

Myocardial depression is a common problem in diseases such as sepsis, and also prognosticates for poor outcomes. Affected patients often require very high doses of current first-line agents (catecholamine inotropes such as adrenaline or dobutamine) as they exhibit decreased responsiveness to these agents. Requirement for high doses of catecholamines is also a poor prognosticator; this may relate to off-target deleterious effects of these agents notwithstanding underlying illness severity.

Hypocholesterolaemia is a recognised marker of severe illness for over 100 years. Multiple studies in sepsis and other critical illnesses (e.g. trauma) show an early drop in cholesterol levels, the magnitude of which prognosticates for a poor outcome.

Currently, treatment for myocardial depression focuses on providing beta-adrenergic agonists such as dobutamine or adrenaline (epinephrine), often at very high doses due to decreased adrenoreceptor sensitivity in myocardial depression patients as described above. Other agents such as levosimendan (calcium channel sensitizer) and enoximone or milrinone (Type III phosphodiesterase inhibitors) are used as adjuncts but have not been demonstrated to show outcome benefit. Control of heart rate with ivabradine (a funny channel inhibitor) has also been tested but not shown in small studies to show any benefit. Studies are currently ongoing with beta-adrenergic blockade as, apart from heart rate control, there is some evidence that beta-blockers may restore adrenergic sensitivity. Such drugs, however, do need to be used cautiously and under close monitoring due to their negative inotropic actions.

As such, there is a need in the field for improved methods of and compositions for treating myocardial depression.

### SUMMARY OF THE INVENTION

The present inventors have discovered that restoration of abnormalities associated with myocardial depression can be achieved by using compositions comprising one or more compounds of formula (II), for example one or more compounds of formula (I). The present inventors have also discovered that compositions comprising one or more compounds of formula (II), for example one or more compounds of formula (I), can be used to increase adrenergic signalling responsiveness.

Accordingly, the present disclosure demonstrates the surprising effect of compounds of formula (II), for example one or more compounds of formula (I), for use as a treatment for myocardial depression and for increasing adrenergic responsiveness in a subject.

Accordingly, the invention provides a method for the treatment of a disease associated with myocardial depression comprising administering to a subject in need thereof a therapeutically effective amount of a composition comprising one or more compounds of formula (II) or a pharmaceutically acceptable salt, hydrate, prodrug or stereoisomer thereof,

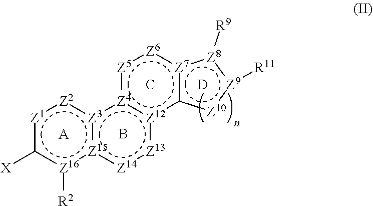

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

In some embodiments, one of Z¹ to Z¹⁶ is NH or N. In other embodiments, two of Z¹ to Z¹⁶ are NH or N. In other embodiments, three of Z¹ to Z¹⁶ are NH or N. In other embodiments, four of Z¹ to Z¹⁶ are NH or N. In other embodiments, none of Z¹ to Z¹⁶ are NH or N.

In some embodiments, n is 0. In other embodiments, n is 1. In other embodiments, n is 2. Preferably, n is 1.

The one or more compounds of formula (II) may be one or more compounds of formula (I) or a pharmaceutically acceptable salt, hydrate, prodrug or stereoisomer thereof,

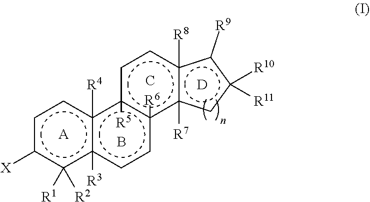

wherein.
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

The invention also provides a method of increasing adrenergic signalling responsiveness in a subject in need thereof, comprising administering to the cell, tissue or subject a therapeutically effective amount of a composition comprising one or more compounds of formula (II) or a pharmaceutically acceptable salt, hydrate, prodrug or stereoisomer thereof,

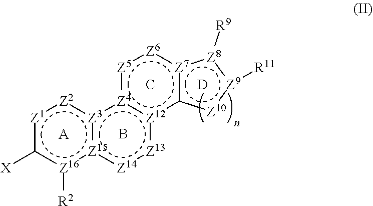

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

In some embodiments, one of Z¹ to Z¹⁶ is NH or N. In other embodiments, two of Z¹ to Z¹⁶ are NH or N. In other embodiments, three of Z¹ to Z¹⁶ are NH or N. In other embodiments, four of Z¹ to Z¹⁶ are NH or N. In other embodiments, none of Z¹ to Z¹⁶ are NH or N.

In some embodiments, n is 0. In other embodiments, n is 1. In other embodiments, n is 2. Preferably, n is 1.

The one or more compounds of formula (II) may be one or more compounds of formula (I) or a pharmaceutically acceptable salt, hydrate, prodrug or stereoisomer thereof,

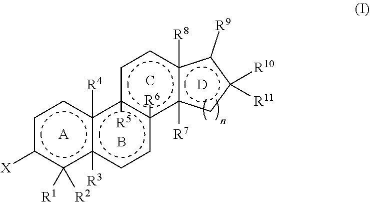

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

The invention also provides a pharmaceutical composition comprising: (i) cholesterol and/or (ii) one or more other compounds with formula (II), or a pharmaceutically acceptable salt, hydrate, prodrug or stereoisomer thereof,

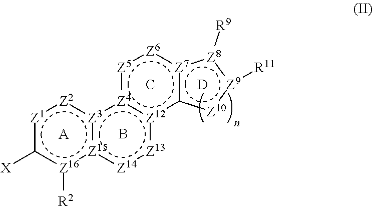

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

In some embodiments, one of Z¹ to Z¹⁶ is NH or N. In other embodiments, two of Z¹ to Z¹⁶ are NH or N. In other embodiments, three of Z¹ to Z¹⁶ are NH or N. In other embodiments, four of Z¹ to Z¹⁶ are NH or N. In other embodiments, none of Z¹ to Z¹⁶ are NH or N.

In some embodiments, n is 0. In other embodiments, n is 1. In other embodiments, n is 2. Preferably, n is 1.

The one or more compounds of formula (II) may be one or more compounds of formula (I) or a pharmaceutically acceptable salt, hydrate, prodrug or stereoisomer thereof,

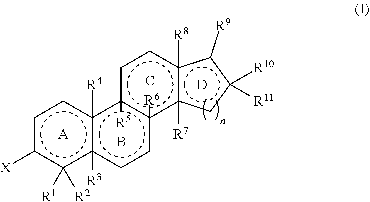

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

The invention also provides a composition comprising one or more compounds with formula (II) or a pharmaceutically acceptable salt, hydrate, prodrug or stereoisomer thereof for use in a method of treatment of a disease associated with myocardial depression, the method comprising administering to a subject in need thereof a therapeutically effective amount of the composition,

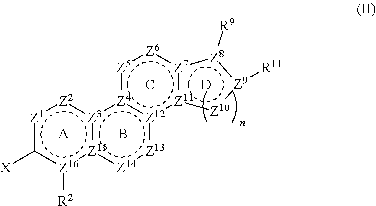

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

In some embodiments, one of Z¹ to Z¹⁶ is NH or N. In other embodiments, two of Z¹ to Z¹⁶ are NH or N. In other embodiments, three of Z¹ to Z¹⁶ are NH or N. In other embodiments, four of Z¹ to Z¹⁶ are NH or N. In other embodiments, none of Z¹ to Z¹⁶ are NH or N.

In some embodiments, n is 0. In other embodiments, n is 1. In other embodiments, n is 2. Preferably, n is 1.

The one or more compounds of formula (II) may be one or more compounds of formula (I) or a pharmaceutically acceptable salt, hydrate, prodrug or stereoisomer thereof,

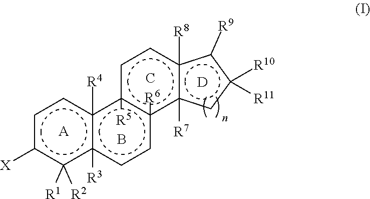

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

The invention also provides a composition comprising one or more compounds with formula (II) or a pharmaceutically acceptable salt, hydrate, prodrug or stereoisomer thereof for use in a method of increasing adrenergic signalling responsiveness in a subject in need thereof, comprising administering to the subject a therapeutically effective amount of the composition,

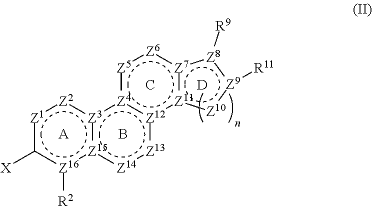

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

In some embodiments, one of Z¹ to Z¹⁶ is NH or N. In other embodiments, two of Z¹ to Z¹⁶ are NH or N. In other embodiments, three of Z¹ to Z¹⁶ are NH or N. In other embodiments, four of Z¹ to Z¹⁶ are NH or N. In other embodiments, none of Z¹ to Z¹⁶ are NH or N.

In some embodiments, n is 0. In other embodiments, n is 1. In other embodiments, n is 2. Preferably, n is 1.

The one or more compounds of formula (II) may be one or more compounds of formula (I) or a pharmaceutically acceptable salt, hydrate, prodrug or stereoisomer thereof,

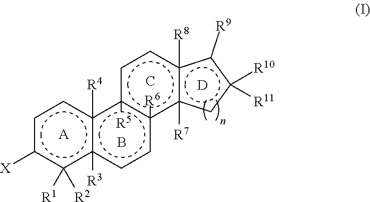

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

### BRIEF DESCRIPTION OF THE FIGURES

FIG. 1. Schema of catecholamine responsiveness study in sham and septic rats.

FIG. 2. Plasma levels of total- and HDL-cholesterol in patients with septic shock. Normal range in adults total cholesterol 4-5 mmol/l, HDL cholesterol 0.93-1.44 mmol/l. The difference between survivor and non-survivor groups was significant at each time point. For each day, for both “Total cholesterol and “HDL cholesterol”, the left-hand bar corresponds to “Survivors” and the right-hand bar corresponds to “Non-survivors”.

FIG. 3. Plasma levels of total- and HDL-cholesterol in rats with faecal peritonitis. N=4 rats per group. For “6 h” and “24 h”, for both “Total cholesterol” and “HDL cholesterol”, the left-hand bar corresponds to “Sham controls”, the middle bar corresponds to “Survivors”, and the right-hand bar corresponds to “Non-survivors”. For “72 h” for both “Total cholesterol” and “HDL cholesterol”, the left-hand bar corresponds to “Sham controls” and the right-hand bar corresponds to “Survivors”.

FIG. 4. Membrane cholesterol concentration in heart and liver cells at 24 h post-sepsis

FIG. 5. In vitro incubation of H9C2 cardiomyocyte cells for 2 h with pooled serum taken at 24 hours from septic or healthy rats.

FIG. 6. Troponin (marker of cardiac injury), B-type natriuretic peptide (marker of ventricular dysfunction) and haemodynamics in patients surviving/not surviving septic shock. *p<0.05. For each of “Troponin T”, “B-type natriuretic peptide”, “stroke volume” and “Heart rate”, the left-hand bar corresponds to “Survivors” and the right-hand bar corresponds to “Non-survivors”.

FIG. 7. Troponin (marker of cardiac injury), B-type natriuretic peptide (marker of ventricular dysfunction) and haemodynamics in sham-operated control, predicted survivors and non-survivors in rat faecal peritonitis model. 6 per group for sham and survivor rats at all points; 8 (6 h); N=5 (24 h) for predicted non-survivors *p<0.05. For each of “Troponin T”, “B-type natriuretic peptide”, “stroke volume” and “Heart rate”, for “6 h” and “24 h”, the left-hand bar corresponds to “Sham controls”, the middle bar corresponds to “Survivors”, and the right-hand bar corresponds to “Non-survivors”. For each of “Troponin”, “B-type natriuretic peptide”, “stroke volume” and “Heart rate”, for “72 h”, the left-hand bar corresponds to “Sham controls” and the right-hand bar corresponds to “Survivors”.

FIG. 8. Plasma catecholamine level in sham-operated control, predicted survivors and non-survivors in rat faecal peritonitis model. N=6 for sham and survivor rats at all points; N=8 (6 h) and N=5 (24 h) for predicted non-survivors rats. *p<0.05. For both “plasma adrenaline” and “plasma noradrenaline”, for “6 h” and “24 h”, the left-hand bar corresponds to “Sham controls”, the middle bar corresponds to “Survivors”, and the right-hand bar corresponds to “Non-survivors”. For both “plasma adrenaline” and “plasma noradrenaline”, for “72 h”, the left-hand bar corresponds to “Sham controls” and the right-hand bar corresponds to “Survivors”.

FIG. 9. Response in stroke volume to dobutamine (circles for “Baseline” and “DOB” on the left for each of the four graphs), and response in blood pressure to noradrenaline (circles for “Baseline” and “NEPI” on the right for each of the four graphs) in sham-operated rats and septic rats at 21 h post-faecal peritonitis insult (n=6-10 per group). *p<0.05

FIG. 10. Percentage change in stroke volume in response to dobutamine, in sham-operated rats and septic rats at 21 h post-faecal peritonitis insult (n=6-10 per group). *p<0.05

FIG. 11. Change in cardiomyocyte membrane cholesterol content in septic animals at 22 hours after treatment with either HDL cholesterol or liposomal cholesterol for 16 hours. *p<0.05

FIG. 12. Change in plasma cholesterol level in septic animals at 22 hours after treatment with either HDL cholesterol or liposomal cholesterol for 16 hours.

FIG. 13. Both liposomal- and HDL-cholesterol reverse hyporeactivity to dobutamine (circles for “Baseline” and “DOB” on the left for each of the eight graphs), but not pressor response to noradrenaline (circles for “Baseline” and “NEPI” on the right for each of the eight graphs), in septic animals after treatment with either HDL cholesterol or liposomal cholesterol for 15 hours (n=6-10 per group). *p<0.05

FIG. 14. Percentage change in stroke volume in response to dobutamine, in septic rats treated with either liposomal- or HDL-cholesterol (n=6-10 per group). *p<0.05

FIG. 15. Restoration of depleted membrane sterol levels in a H9C2 cardiomyocyte cell line following incubation cholesterol or sitosterol. n=3 for all groups *p<0.05

FIG. 16. Sterol treatment increases adrenergic signalling in cardiomyocytes. N=4 for MbCD/MbCD-Chol/AD and N=5 for MbCD/MbCD-Sito/control groups. *p<0.05

FIG. 17. H9C2 cardiomyocyte sterol levels depleted with methyl-ß-cyclodextrin over 30 mins can be restored to normal (at 75 μM) or supranormal (at 100 μM) levels by addition of different pegylated liposomes containing a cholesterol-sitosterol mixture for one hour.

FIG. 18. H9C2 cardiomyocyte sterol levels depleted with methyl-ß-cyclodextrin over 30 mins can be increased to normal (at 100 μM) levels by addition of non-pegylated liposomes containing stigmasterol alone for one hour.

FIG. 19. Liposomes carrying cholesterol and phytosterol rescue dobutamine (adrenergic) signalling in H9C2 cardiomyocytes following cholesterol depletion with methyl-ß-cyclodextrin (MßCD)

### DETAILED DESCRIPTION

The present invention is concerned with methods of treating myocardial depression or increasing adrenergic signalling responsiveness in a subject, by administering to a subject in need thereof a therapeutically effective amount of a composition comprising one or more compounds of formula (II), for example a composition comprising one or more compounds of formula (I).

The present invention is also concerned with compositions comprising one or more compounds of formula (II), for example compositions comprising one or more compounds of formula (I), for use in methods of treating myocardial depression or increasing adrenergic signalling responsiveness in a subject.

The present invention also relates to pharmaceutical compositions comprising a blend of cholesterol and one or more other compounds of formula (II), for example one or more other compounds of formula (I).

### Definitions

As defined herein, the term “alkyl” refers to a linear or branched saturated monovalent hydrocarbon radical having the number of carbon atoms indicated in the prefix. Thus, the term “C₁₋₄ alkyl” refers to a linear saturated monovalent hydrocarbon radical of one to four carbon atoms or a branched saturated monovalent hydrocarbon radical of three or four carbon atoms, e.g. methyl, ethyl, n-propyl, iso-propyl, n-butyl, iso-butyl and tert-butyl. Preferably an alkyl group is a C₁₋₂₀ alkyl group, more preferably a C₁₋₁₂ alkyl group and even more preferably a C₁₋₈ alkyl group, e.g. a C₁₋₆ alkyl group.

As used herein, the term “alkenyl” refers to a linear or branched unsaturated monovalent hydrocarbon radical having the number of carbon atoms indicated in the prefix and containing at least one double bond. Thus, the term “C₂₋₆ alkenyl” refers to a linear unsaturated monovalent hydrocarbon radical of two to six carbon atoms having at least one double bond, or a branched unsaturated monovalent hydrocarbon radical of three to six carbon atoms having at least one double bond, e.g. ethenyl, propenyl, 1,3-butadienyl, (CH₂)₂CH═C(CH₃)₂, CH₂CH═CHCH(CH₃)₂, and the like. The number of double bonds in an alkenyl group is not particularly limited, e.g. an alkenyl group may contain one, two, three or more double bonds. Preferably an alkenyl group is a C₂₋₂₀ alkenyl group, more preferably a C₂₋₁₂ alkenyl group and even more preferably a C₂₋₈ alkenyl group, e.g. a C₂₋₆ alkenyl group.

As used herein, the term “alkynyl” refers to a linear or branched unsaturated monovalent hydrocarbon radical having the number of carbon atoms indicated in the prefix and containing at least one triple bond. Thus, the term “C₂₋₆ alkynyl” refers to a linear unsaturated monovalent hydrocarbon radical of two to six carbon atoms having at least one triple bond, or a branched unsaturated monovalent hydrocarbon radical of four to six carbon atoms having at least one double bond, e.g. ethynyl, propynyl, and the like. The number of triple bonds in an alkynyl group is not particularly limited, e.g. an alkenyl group may contain one, two, three or more triple bonds. Preferably an alkenyl group is a C₂₋₂₀ alkynyl group, more preferably a C₂₋₁₂ alkynyl group and even more preferably a C₂₋₈ alkynyl group, e.g. a C₂₋₆ alkynyl group.

As used herein, the term “alkylene” refers to a linear saturated divalent hydrocarbon radical or a branched saturated divalent hydrocarbon radical having the number of carbon atoms indicated in the prefix, e.g. methylene, ethylene, propylene, 1-methylpropylene, 2-methylpropylene, butylene, pentylene, and the like. Preferably, an alkylene group is a C₁₋₂₀ alkylene group, more preferably a C₁₋₁₂ alkylene group and even more preferably a C₁₋₈ alkylene group, e.g. a C₁₋₆ alkylene group.

As defined herein, the term “cycloalkane” refers to a cyclic saturated hydrocarbon moiety having the number of carbon atoms indicated in the prefix. Thus, the term “C₅₋₇ cycloalkane” refers to a cyclic saturated hydrocarbon moiety having five to seven carbon atoms, e.g. cyclopentane, cyclohexane, or cycloheptane. Preferably a cycloalkane group is a C₅₋₇ cycloalkane group.

As defined herein, the term “acyl” refers to a —COR radical, wherein R is alkyl, haloalkyl, cycloalkyl, cycloalkylalkyl, aryl, aralkyl, heteroaryl, heteroaralkyl, heterocyclyl, or heterocyclylalkyl, each as defined herein, or poly(ethylene glycol), and wherein R is optionally further substituted with one, two, three, four or more substituents independently selected from alkyl, alkoxy, halo, haloalkoxy, —OH, —NH₂, alkylamino, —COOH, or alkoxycarbonyl.

As defined herein, the term “alkoxy” refers to an —OR radical where R is alkyl as defined above, e.g., methoxy, ethoxy, n-propoxy, iso-propoxy, n-butoxy, iso-butoxy, tert-butoxy and the like. Preferably an alkoxy group is a C₁₋₂₀ alkoxy group, more preferably a C₁₋₁₂ alkoxy group and even more preferably a C₁₋₆ alkoxy group.

As defined herein, the term “alkoxycarbonyl” or “ester” refers to a —C(O)OR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, or poly(ethylene glycol), and wherein R is optionally further substituted with one, two, three, four or more substituents independently selected from alkyl, alkoxy, halo, haloalkoxy, —OH, —NH₂, alkylamino, —COOH, or alkoxycarbonyl.

As defined herein, the term “alkylamino” refers to an —NHR radical where R is alkyl as defined above, e.g. methylamino, ethylamino, n-propylamino, iso-propylamino, and the like. Preferably an alkylamino group is a C₁₋₂₀ alkylamino group, more preferably a C₁₋₁₂ alkylamino group and even more preferably a C₁₋₆ alkylamino group.

As defined herein, the term “aryl” refers to a monovalent monocyclic or bicyclic aromatic hydrocarbon radical of 6 to 10 ring atoms, e.g. phenyl or naphthyl, and the like.

As defined herein, the term “aralkyl” refers to an -(alkylene)-R radical where R is aryl as defined above.

As defined herein, the term “carbamyl” refers to a —C(O)NR^{x}R^{y} radical where R^{x} and R^{y} are independently hydrogen, alkyl, haloalkyl, cycloalkyl, cycloalkylalkyl, aryl, aralkyl, heteroaryl, heteroaralkyl, heterocyclyl, or heterocyclylalkyl, each as defined herein, or poly(ethylene glycol), and wherein R^{x} and R^{y} are optionally further substituted with one, two, three, four or more substituents independently selected from alkyl, alkoxy, halo, haloalkoxy, —OH, —NH₂, alkylamino, —COOH, or alkoxycarbonyl.

As defined herein, the term “cycloalkyl” refers to a cyclic saturated monovalent hydrocarbon radical of three to ten carbon atoms wherein one or two carbon atoms may be replaced by an oxo group, e.g. cyclopropyl, cyclobutyl, cyclopentyl, or cyclohexyl, and the like. Preferably a cycloalkyl group is a C₃₋₁₂ cycloalkyl group, more preferably a C₃₋₈ cycloalkyl group and even more preferably a C₃₋₆ cycloalkyl group.

As defined herein, the term “cycloalkylalkyl” refers to an -(alkylene)-R radical where R is cycloalkyl as defined above, e.g. cyclopropylmethyl, cyclobutylmethyl, cyclopentylethyl, or cyclohexylmethyl, and the like.

As defined herein, the term “halo” refers to fluoro, chloro, bromo, or iodo, preferably fluoro or chloro.

As defined herein, the term “haloalkyl” refers to an alkyl radical as defined above, which is substituted with one or more halogen atoms, preferably one to five halogen atoms, preferably fluorine or chlorine, including those substituted with different halogens, e.g. —CH₂Cl, —CF₃, —CHF₂, —CH₂CF₃, —CF₂CF₃, —CF(CH₃)₂, and the like. Preferably a haloalkyl group is a C₁₋₂₀ haloalkyl group, more preferably a C₁₋₁₂ haloalkyl group and even more preferably a C₁₋₆ haloalkyl group.

As defined herein, the term “haloalkoxy” refers to an —OR radical where R is haloalkyl as defined above, e.g. —OCF₃, —OCHF₂, and the like. Preferably a haloalkoxy group is a C₁₋₂₀ haloalkoxy group, more preferably a C₁₋₁₂ haloalkoxy group and even more preferably a C₁₋₆ haloalkoxy group.

As defined herein, the term “heteroaryl” refers to a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

As defined herein, the term “heteroaralkyl” refers to an -(alkylene)-R radical where R is heteroaryl as defined above.

As defined herein, the term “heterocycyl” refers to a saturated or unsaturated monovalent monocyclic group of 4 to 8 ring atoms in which one or two ring atoms are heteroatoms selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds, provided that the ring is not aromatic.

As defined herein, the term “heterocycloalkyl” refers to an -(alkylene)-R radical where R is heterocyclyl ring as defined above, e.g. tetraydrofuranylmethyl, piperazinylmethyl, morpholinylethyl, and the like.

Typically, said “optionally substituted” moieties in a compound of Formula (II), i.e. said optionally substituted C₁₋₂₀ alkyl, C₂₋₂₀ alkenyl and C₂₋₂₀ alkynyl groups, are each independently optionally substituted with a moiety that is uncharged at physiological pH (i.e. pH 7.5). Thus, typically said optionally substituted C₁₋₂₀ alkyl, C₂₋₂₀ alkenyl and C₂₋₂₀ alkynyl groups are each independently optionally substituted with one, two, three, four, five or six groups selected from C₁₋₂₀ alkyl, C₁₋₂₀ alkenyl, C₂₋₂₀ alkynyl, C₁₋₂₀ alkoxy, C₅₋₇ cycloalkyl, C₆₋₁₀ aryl, C₇₋₂₀ aralkyl, C₁₋₂₀ acyl, halo, C₁₋₂₀ haloalkyl or C₁₋₂₀ haloalkoxy. Preferably, said optionally substituted groups are independently optionally substituted with one, two, three, four, five or six groups selected from C₁₋₁₂ alkyl, C₁₋₁₂ alkenyl, C₂₋₁₂ alkynyl, C₁₋₁₂ alkoxy, C₅₋₇ cycloalkyl, C₆₋₁₀ aryl, C₇₋₁₂ aralkyl, C₁₋₁₂ acyl, halo, C₁₋₁₂ haloalkyl or C₁₋₁₂ haloalkoxy. More preferably, said optionally substituted groups are independently optionally substituted with one, two, three, four, five or six groups selected from C₁₋₆ alkyl, C₁₋₆ alkenyl, C₂₋₆ alkynyl, C₁₋₆ alkoxy, C₅₋₇ cycloalkyl, C₆₋₁₀ aryl, C₇₋₁₂ aralkyl, C₁₋₆ acyl, halo, C₁₋₆ haloalkyl or C₁₋₆ haloalkoxy. Preferably, said optionally substituted groups are independently optionally substituted with one, two, three or four such groups, more preferably with one or two such groups, and most preferably with one such group.

As defined herein, the term “stereoisomer” refers to a compound made up of the same atoms bonded by the same bonds but having different three-dimensional structures, which are not interchangeable. As defined herein, the term “enantiomer” refers to one of a pair of stereoisomers whose molecules are non-superimposable mirror images of one another. As defined herein, the term “racemate” refers to a 50:50 mixture of an pair of enantiomers. As defined herein, the term “diastereoisomer” refers to one of a pair (or more) of stereoisomers that have at least two stereogenic centres, but which are not mirror images of each other.

As defined herein, the term “pharmaceutically acceptable salt” of a given compound refers to salts that retain the biological effectiveness and properties of the given compound and which are not biologically or otherwise undesirable. “Pharmaceutically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, e.g. isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

As defined herein, the term “hydrate” refers to the complex formed by the combining of a compound described herein and water.

As defined herein, the term “prodrug” of a compound of the disclosure refers to any compound or pharmaceutically acceptable salt thereof which, after administration to the human body, may be metabolised in vivo to a compound of the disclosure. Typical prodrugs include acyl, ester and carbamyl derivatives of the compound of formula (II), for example acyl, ester and carbamyl derivatives of the compound of formula (I), wherein the acyl, ester or carbamyl moiety is directly bonded to a heteroatom in moiety X. In particular, when X in formula (II) is OH, a typical prodrug of formula (II) is a compound of formula (II) in which X is O-acyl, O-ester or O-carbamyl. For example, when X is formula (I) is OH, a typical prodrug of formula (I) is a compound of formula (I) in which X is O-acyl, O-ester or O-carbamyl. Similarly, when X in formula (II) is NH₂ or NHR, a typical prodrug of formula (II) is a compound of formula (II) in which X is NH-acyl, NR-acyl, NH-ester, NR-ester, NH-carbamyl or NR-carbamyl. For example, when X in formula (I) is NH₂ or NHR, a typical prodrug of formula (I) is a compound of formula (I) in which X is NH-acyl, NR-acyl, NH-ester, NR-ester, NH-carbamyl or NR-carbamyl.

### Compounds Useful in the Invention

The one or more compounds useful in the present invention are each independently a compound of formula (II):

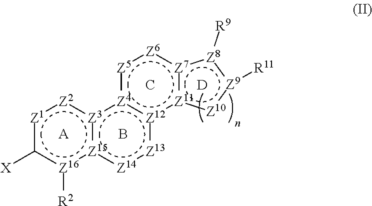

or a pharmaceutically acceptable salt, hydrate, prodrug or stereoisomer thereof, wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

In some embodiments, one of Z¹ to Z¹⁶ is NH or N. In other embodiments, two of Z¹ to Z¹⁶ are NH or N. In other embodiments, three of Z¹ to Z¹⁶ are NH or N. In other embodiments, four of Z¹ to Z¹⁶ are NH or N. In other embodiments, none of Z to Z¹⁶ are NH or N.

The one or more compounds useful in the present invention may each independently be a compound of formula (I):

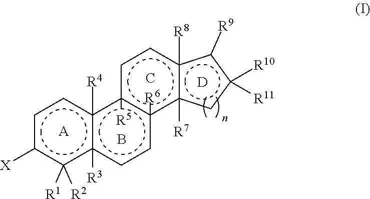

or a pharmaceutically acceptable salt, hydrate, prodrug or stereoisomer thereof, wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

Preferred embodiments of the compound of formula (II) or formula (I), or pharmaceutically acceptable salts, hydrates, prodrugs or stereoisomers thereof, are set out below.

X is preferably selected from OH, NH₂, CO₂H and SO₂H, and is more preferably OH or CO₂H, and is most preferably OH.

R¹ is preferably hydrogen or C₁₋₄ alkyl, more preferably hydrogen, methyl or ethyl, still more preferably hydrogen or methyl, and most preferably hydrogen.

R² is typically present. Alternatively, though, R² may be absent. R² is preferably hydrogen or C₁₋₄ alkyl, more preferably hydrogen, methyl or ethyl, still more preferably hydrogen or methyl, and most preferably hydrogen.

Typically, R¹ and R² are the same. Thus, in some embodiments, both R¹ and R² are hydrogen. Alternatively, both R¹ and R² are C₁₋₆ alkyl, preferably C₁₋₄ alkyl, more preferably methyl or ethyl, and most preferably methyl. Alternatively, R¹ and R² are different. Thus, in some embodiments, R¹ is C₁₋₆ alkyl, preferably C₁₋₄ alkyl, more preferably methyl or ethyl, and most preferably methyl, and R² is hydrogen.

In some embodiments, R³ is present. In other embodiments, R³ is absent. If present, R³ is preferably hydrogen or C₁₋₄ alkyl, more preferably hydrogen, methyl or ethyl, still more preferably hydrogen or methyl, and most preferably hydrogen. Alternatively, in some preferable embodiments, R³ is absent.

R⁴ is typically present. Alternatively, though, R⁴ may be absent. In a preferable embodiment, R⁴ is present and is hydrogen or C₁₋₄ alkyl, more preferably hydrogen, methyl or ethyl, still more preferably hydrogen or methyl, and most preferably hydrogen. In said embodiment, R⁵ is preferably hydrogen or C₁₋₄ alkyl, more preferably hydrogen, methyl or ethyl, still more preferably hydrogen or methyl, and most preferably hydrogen. In an alternative preferred embodiment, in the compound of Formula (I), R⁴ and R⁵ together with the adjacent carbons in ring B form unsubstituted cyclopropane.

R⁵ is typically present. Alternatively, though, R⁵ may be absent. R⁵ is preferably hydrogen or C₁₋₄ alkyl, more preferably hydrogen, methyl or ethyl, still more preferably hydrogen or methyl, and most preferably hydrogen.

In some embodiments, R⁶ is present. In other embodiments, R⁶ is absent. R⁶ is preferably hydrogen or C₁₋₄ alkyl, more preferably hydrogen, methyl or ethyl, still more preferably hydrogen or methyl, and most preferably hydrogen. Alternatively, in some preferable embodiments, R⁶ is absent.

R⁷ is typically present. Alternatively, though, R⁷ may be absent. R⁷ is preferably hydrogen or C₁₋₄ alkyl, more preferably hydrogen, methyl or ethyl, still more preferably hydrogen or methyl, and most preferably hydrogen.

R⁸ is typically present. Alternatively, though, R⁸ may be absent. R⁸ is preferably hydrogen or C₁₋₄ alkyl, more preferably hydrogen, methyl or ethyl, still more preferably hydrogen or methyl, and most preferably hydrogen.

In a preferred embodiment, R⁹ is represented by the formula

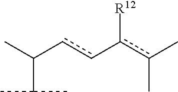

wherein:
- - - - represents the point of attachment to ring D;
each [symbol] independently represents a single or a double bond; and
R¹² is selected from hydrogen or C₁₋₄ alkyl.

In this embodiment, each [symbol] typically represents a single bond. Alternatively in this embodiment, each [symbol] represents a double bond. Alternatively, one [symbol] represents a single bond and the other a double bond.

In this embodiment, R¹² is preferably hydrogen, methyl or ethyl. Thus, typically, R¹² is hydrogen. Alternatively, R¹² is methyl. Alternatively, R¹² is ethyl.

In this embodiment, R¹⁰ is hydrogen or C₁₋₆ alkyl, preferably hydrogen or C₁₋₄ alkyl, more preferably hydrogen, methyl or ethyl, yet more preferably hydrogen or methyl, and is most preferably hydrogen.

In an alternative embodiment, in the compound of Formula (I) R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl. Preferably, R⁹ and R¹⁰ together with the adjacent carbons in ring D form a cyclopentane or cyclohexane ring wherein the carbon atoms that do not also form part of ring D may be optionally substituted with at least one C₁₋₆ alkyl, C₂₋₆ alkenyl or C₂₋₆ alkynyl. More preferably, R⁹ and R¹⁰ together with the adjacent carbons in ring D form a cyclopentane ring wherein the carbon atoms that do not also form part of ring D may be optionally substituted with at least one C₁₋₆ alkyl, C₂₋₆ alkenyl or C₂₋₆ alkynyl. The carbon atoms in the cylcoalkane (e.g. cyclopentane or cyclohexane) moiety that do not also form part of ring D may in total optionally be substituted with one, two, three or four C₁₋₆ alkyl, C₂₋₆ alkenyl or C₂₋₆ alkynyl, more preferably one or two C₁₋₆ alkyl, C₂₋₆ alkenyl or C₂₋₆ alkynyl, and most preferably one C₁₋₆ alkyl, C₂₋₆ alkenyl or C₂₋₆ alkynyl. A particularly preferred substituent is a C₂₋₄ alykenyl group, such as a vinyl, 1-allyl or 2-allyl group. The most preferred substituent is a 2-allyl group.

R¹¹ is typically present. Alternatively, though, R¹¹ may be absent. R¹¹ is preferably hydrogen or C₁₋₄ alkyl, more preferably hydrogen, methyl or ethyl, still more preferably hydrogen or methyl, and most preferably hydrogen.

In some embodiments, n is 0. Alternatively, n is 1. Alternatively, n is 2. Preferably, n is 1.

Typically, each of rings A, B, C and D in formula (I) independently contain 0, 1, 2 or 3 double bonds. Each ring may independently be fully saturated, partially unsaturated, or fully unsaturated. Each ring may independently be aromatic or non-aromatic. In some embodiments, two or more of rings A, B, C and D together form an aromatic system. Preferably, ring A contains 0 internal double bonds. Preferably, ring B contains 0, 1 or 2 internal double bonds. Preferably, ring C contains 0 internal double bonds. Preferably, ring D contains 0 internal double bonds. Thus, preferably, the compound of formula (I) is a compound of formula (Ia):

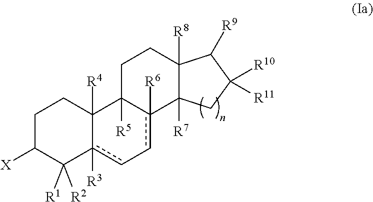

or a pharmaceutically acceptable salt, hydrate, prodrug or stereoisomer thereof, wherein:
X, n and R¹ to R¹¹ are as defined above; and
each [symbol] independently represents a single or a double bond.

Typically, in the compound of formula (Ia), no double bonds are present in the fused ring core, i.e. each [symbol] independently represents a single bond and both R³ and R⁶ are present. Alternatively, in the compound of formula (Ia), one double bond is present in the fused ring core, i.e. one [symbol] represents a single bond and the other [symbol] represents a double bond, and one of R³ and R⁶ is present and the other is absent. Alternatively, in the compound of formula (Ia), two double bonds are present in the fused ring core, i.e. each [symbol] independently represents a double bond, and both R³ and R⁶ are absent.

It will be appreciated that the fused ring system in compounds of formula (I) and (Ia) contains multiple stereogenic centres. The compounds of formula (I) and (Ia) can therefore be isolated as single stereoisomers, or as mixtures of stereoisomers. For instance, the compounds of formula (I) and (Ia) can be isolated as single diastereomers. It is well-known in the art how to separate diastereomers, e.g. by stationary bed chromatography, simulated moving bed chromatography or HPLC. The compounds of formula (I) may also be optically active or racemic forms. It is well-known in the art how to prepare optically active forms, such as by resolution of materials. For the avoidance of doubt, formula (I) and formula (Ia) encompass all stereoisomeric forms of the compounds, including all diasteromers, enantiomeric and racemic forms of the compounds, as well as all mixtures of enantiomers and/or diastereomers of the compounds. Likewise, if the compounds of formula (I) and (Ia) contain olefinic bonds which are external to the fused ring system, unless specified otherwise, it is intended that the formulae encompass both the (E) and (Z) geometric isomeric forms, and any mixture thereof.

Preferably, the compound of formula (Ia) is a compound of formula (Ib):

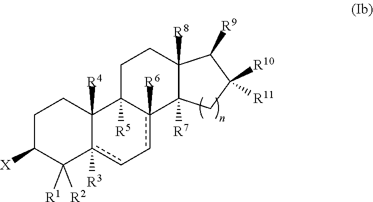

or a pharmaceutically acceptable salt, hydrate, prodrug or optical isomer thereof, wherein:
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl;
R³ is selected from hydrogen and C₁₋₆ alkyl, or may be absent;
R⁴ is selected from hydrogen and C₁₋₆ alkyl;
R⁵ is selected from hydrogen and C₁₋₆ alkyl;
R⁶ is selected from hydrogen and C₁₋₆ alkyl, or may be absent;
R⁷ is selected from hydrogen and C₁₋₆ alkyl;
R⁸ is selected from hydrogen and C₁₋₆ alkyl;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2; and
each [symbol] independently represents a single or a double bond.

X is preferably selected from OH, NH₂, CO₂H and SO₂H, and is more preferably OH or CO₂H, and is most preferably OH.

R¹ is preferably hydrogen or C₁₋₄ alkyl, more preferably hydrogen, methyl or ethyl, still more preferably hydrogen or methyl, and most preferably hydrogen.

R² is preferably hydrogen or C₁₋₄ alkyl, more preferably hydrogen, methyl or ethyl, still more preferably hydrogen or methyl, and most preferably hydrogen.

R³ is preferably hydrogen or C₁₋₄ alkyl, more preferably hydrogen, methyl or ethyl, still more preferably hydrogen or methyl, and most preferably hydrogen. Alternatively, in some preferable embodiments, R³ is absent.

R⁴ is preferably hydrogen or C₁₋₄ alkyl, more preferably hydrogen, methyl or ethyl, still more preferably hydrogen or methyl, and most preferably hydrogen.

R⁵ is preferably hydrogen or C₁₋₄ alkyl, more preferably hydrogen, methyl or ethyl, still more preferably hydrogen or methyl, and most preferably hydrogen.

R⁵ is preferably hydrogen or C₁₋₄ alkyl, more preferably hydrogen, methyl or ethyl, still more preferably hydrogen or methyl, and most preferably hydrogen.

R⁶ is preferably hydrogen or C₁₋₄ alkyl, more preferably hydrogen, methyl or ethyl, still more preferably hydrogen or methyl, and most preferably hydrogen. Alternatively, in some preferable embodiments, R⁶ is absent.

R⁷ is preferably hydrogen or C₁₋₄ alkyl, more preferably hydrogen, methyl or ethyl, still more preferably hydrogen or methyl, and most preferably hydrogen.

R⁸ is preferably hydrogen or C₁₋₄ alkyl, more preferably hydrogen, methyl or ethyl, still more preferably hydrogen or methyl, and most preferably hydrogen.

In a preferred embodiment, R⁹ is represented by the formula

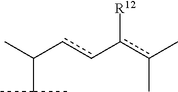

wherein:
- - - - represents the point of attachment to ring D;
each [symbol] independently represents a single or a double bond; and
R¹² is selected from hydrogen or C₁₋₄ alkyl.

In this embodiment, [symbol] typically represents a single bond. Alternatively in this embodiment, [symbol] represents a double bond. Alternatively, one [symbol] represents a single bond and the other a double bond.

In this embodiment, R¹² is preferably hydrogen, methyl or ethyl. Thus, typically, R¹² is hydrogen. Alternatively, R¹² is methyl. Alternatively, R¹² is ethyl.

In this embodiment, R¹⁰ is hydrogen or C₁₋₆ alkyl, preferably hydrogen or C₁₋₄ alkyl, more preferably hydrogen, methyl or ethyl, yet more preferably hydrogen or methyl, and is most preferably hydrogen.

In this embodiment, R⁹ may be represented by the formula

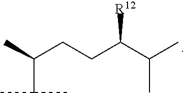

Alternatively, in this embodiment, R⁹ may be represented by the formula

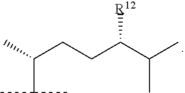

Alternatively, in this embodiment, R⁹ may be represented by the formula

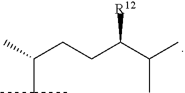

Alternatively, in this embodiment, R⁹ may be represented by the formula

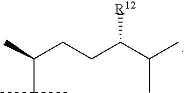

Alternatively, in this embodiment, R⁹ may be represented by the formula

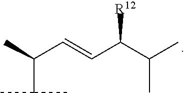

Alternatively, in this embodiment, R⁹ may be represented by the formula

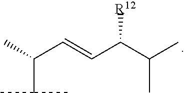

Alternatively, in this embodiment, R⁹ may be represented by the formula

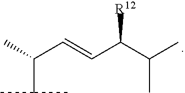

Alternatively, in this embodiment, R⁹ may be represented by the formula

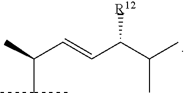

Alternatively, in this embodiment, R⁹ may be represented by the formula

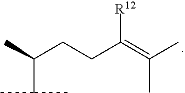

Alternatively, in this embodiment, R⁹ may be represented by the formula

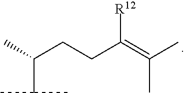

Alternatively, in this embodiment, R⁹ may be represented by the formula

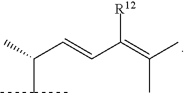

Alternatively, in this embodiment, R⁹ may be represented by the formula

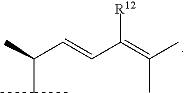

In an alternative embodiment, R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl. Preferably, R⁹ and R¹⁰ together with the adjacent carbons in ring D form a cyclopentane or cyclohexane ring wherein the carbon atoms that do not also form part of ring D may be optionally substituted with at least one C₁₋₆ alkyl, C₂₋₆ alkenyl or C₂₋₆ alkynyl. More preferably, R⁹ and R¹⁰ together with the adjacent carbons in ring D form a cyclopentane ring wherein the carbon atoms that do not also form part of ring D may be optionally substituted with at least one C₁₋₆ alkyl, C₂₋₆ alkenyl or C₂₋₆ alkynyl. The carbon atoms in the cylcoalkane (e.g. cyclopentane or cyclohexane) moiety that do not also form part of ring D may in total optionally be substituted with one, two, three or four C₁₋₆ alkyl, C₂₋₆ alkenyl or C₂₋₆ alkynyl, more preferably one or two C₁₋₆ alkyl, C₂₋₆ alkenyl or C₂₋₆ alkynyl, and most preferably one C₁₋₆ alkyl, C₂₋₆ alkenyl or C₂₋₆ alkynyl. A particularly preferred substituent is a C₂₋₄ alykenyl group, such as a vinyl, 1-allyl or 2-allyl group. The most preferred substituent is a 2-allyl group.

R¹¹ is preferably hydrogen or C₁₋₄ alkyl, more preferably hydrogen, methyl or ethyl, still more preferably hydrogen or methyl, and most preferably hydrogen.

In some embodiments, n is 0. Alternatively, n is 1. Alternatively, n is 2. Preferably, n is 1.

Typically, in the compound of formula (Ib), no double bonds are present in the fused ring core, i.e. each [symbol] independently represents a single bond and both R³ and R⁶ are present. Alternatively, in the compound of formula (Ib), one double bond is present in the fused ring core, i.e. one [symbol] represents a single bond and the other [symbol] represents a double bond, and one of R³ and R⁶ is present and the other is absent. Alternatively, in the compound of formula (Ib), two double bonds are present in the fused ring core, i.e. each [symbol] independently represents a double bond, and both R³ and R⁶ are absent. Thus, a compound of formula (Ib) may be of formula (Ic), formula (Id) or formula (Ie):

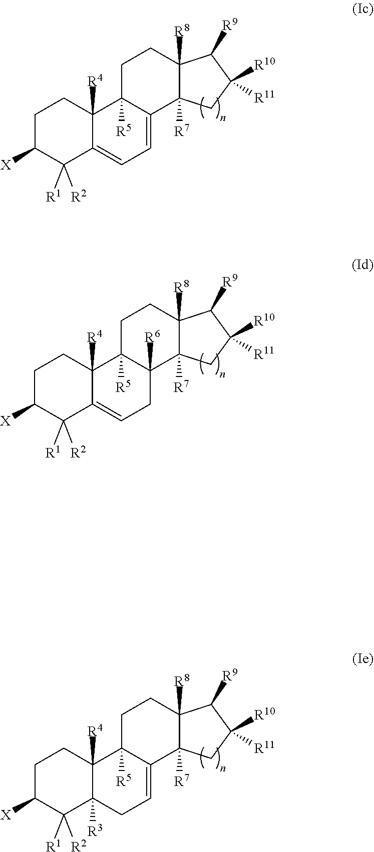

or a pharmaceutically acceptable salt, hydrate, prodrug or optical isomer thereof, wherein X, n and R¹ to R¹¹ are as defined above for formula (Ib).

In preferred embodiments, the compound is a compound of formula (Ic), (Id) or (Ie) wherein:
X is selected from OH, NH₂, NHR, CO₂H, CO₂R, SO₂H and SO₂R, wherein each R is independently C₁₋₆ alkyl, preferably wherein X is selected from OH, NH₂, CO₂H and SO₂H, and most preferably wherein X is OH;
R¹ is hydrogen or methyl;
R² is hydrogen or methyl;
R³ when present, is hydrogen or methyl;
R⁴ is hydrogen or methyl;
R⁵ is hydrogen or methyl;
R⁶, when present, is hydrogen or methyl;
R⁷ is hydrogen or methyl;
R⁸ is hydrogen or methyl;
R⁹ is represented by the formula

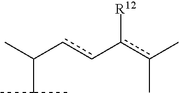

wherein:
   - - - - represents the point of attachment to ring D;
   each [symbol] independently represents a single or a double bond; and
   R¹² is selected from hydrogen, methyl or ethyl;
R¹⁰ is hydrogen or methyl, more preferably hydrogen;
R¹¹ is hydrogen or methyl, more preferably hydrogen; and
n is 0, 1 or 2.

In one particularly preferred embodiment, X is OH, n is 1, R¹, R², R³ (if present), R⁵, R⁶ (if present), R⁷, R¹⁰ and R¹¹ are hydrogen, R⁴ and R⁸ are methyl, and R⁹ is represented by the formula

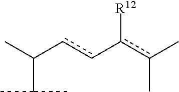

wherein:
- - - - represents the point of attachment to ring D;
each [symbol] independently represents a single or a double bond; and
   R¹² is selected from hydrogen, methyl or ethyl.

In an alternative particularly preferred embodiment, X is OH, n is 1, R³ (if present), R⁵, R⁶ (if present), R⁷, R¹⁰ and R¹¹ are hydrogen, R¹, R², R⁴ and R⁸ are methyl, and R⁹ is represented by the formula

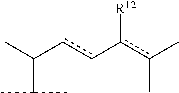

wherein:
- - - - represents the point of attachment to ring D;
each [symbol] independently represents a single or a double bond; and
R¹² is selected from hydrogen, methyl or ethyl.

Thus, in any of the foregoing embodiments, R⁹ may be represented by one of the following formulae:

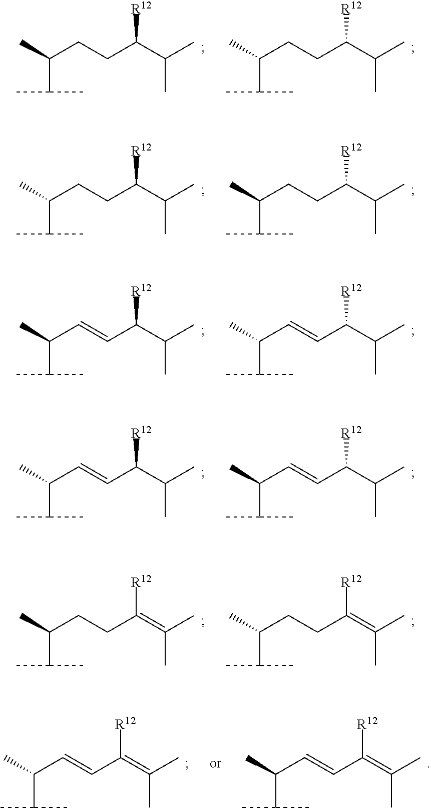

Typically, R¹² is methyl. Alternatively, R¹² is ethyl. Alternatively, R¹² is hydrogen. Preferably, R¹² is methyl or ethyl.

In particularly preferred embodiments, n is 1. Thus, a compound of formula (Ib) may be of formula (If), formula (Ig) or formula (Ih):

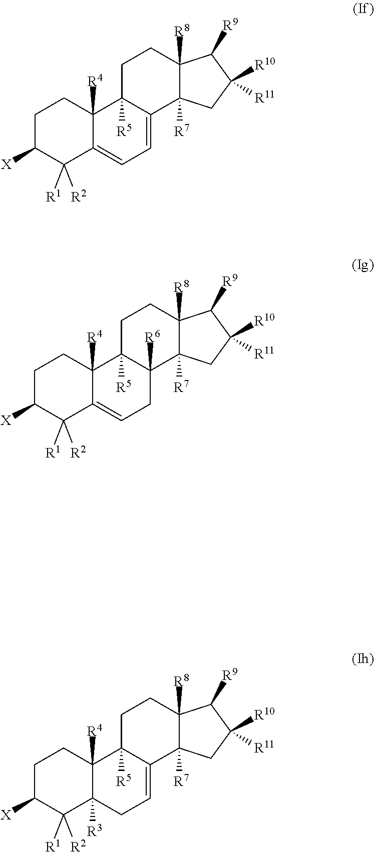

or a pharmaceutically acceptable salt, hydrate, prodrug or optical isomer thereof, wherein X and R¹ to R¹¹ are as defined above for any of formulae (Ib), (Ic), (Id) or (Ie).

In particularly preferred embodiments, a compound of formula (Ib) may be of formula (Ij), (Ik) or (Il):

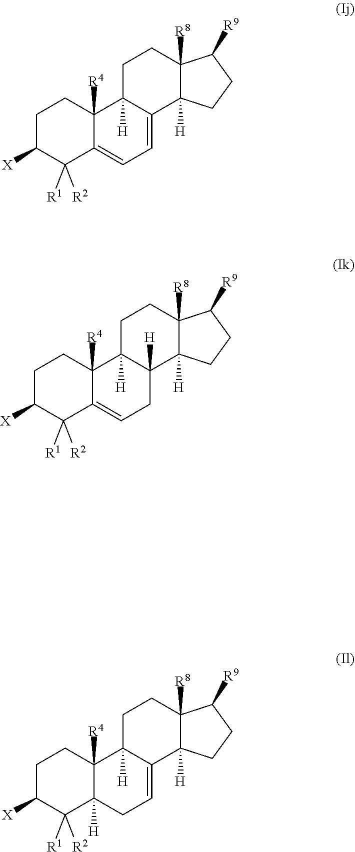

or a pharmaceutically acceptable salt, hydrate, prodrug or optical isomer thereof, wherein X, R¹,
R², R⁴, R⁸ and R⁹ are as defined above for any of formulae (Ib), (Ic), (Id) or (Ie).

In a particularly preferred embodiment, the compound of formula (I) is a compound of formula (Ik). Preferably in this embodiment, X is OH. Preferably in this embodiment, R¹ is hydrogen. Preferably in this embodiment, R² is hydrogen. Preferably in this embodiment, R⁴ is methyl. Preferably in this embodiment, R⁸ is methyl. Preferably in this embodiment, R⁹ is

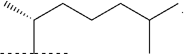

Most preferably in this embodiment, X is OH, R¹ is hydrogen, R² is hydrogen, R⁴ is methyl, R⁸ is methyl and R⁹ is

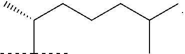

Alternatively, in a compound of formula (I), R⁴ and R⁵ together with the adjacent carbons in ring B form cyclopropane optionally substituted on the carbon which does not form part of ring B with at least one C₁₋₆ alkyl. Thus, the compound of formula (I) may be a compound of formula (Im):

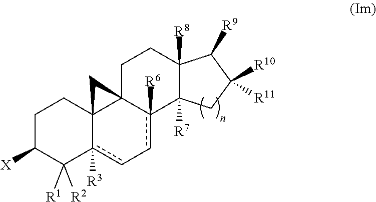

or a pharmaceutically acceptable salt, hydrate, prodrug or optical isomer thereof, wherein X, n, R¹ to R³ and R⁶ to R¹¹ are as defined above for any of formulae (Ib), (Ic), (Id) or (Ie).

Most preferably, the compound of formula (I) is selected from:

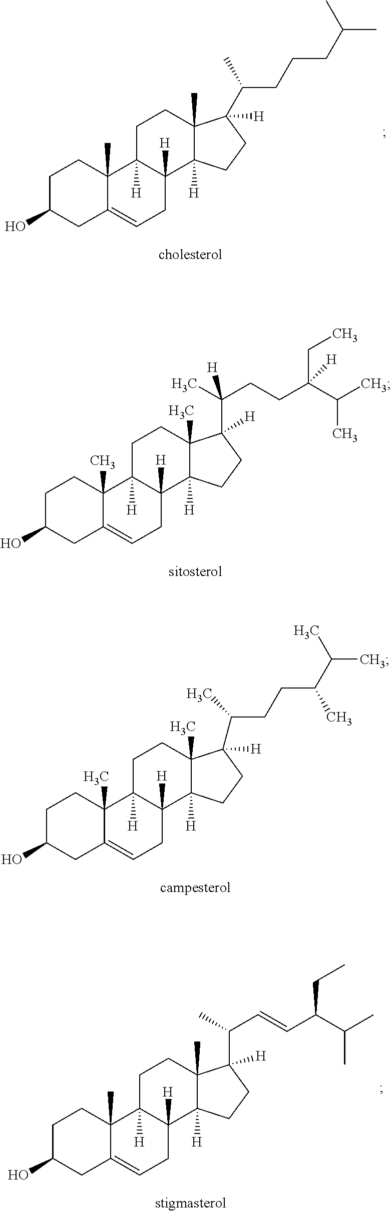

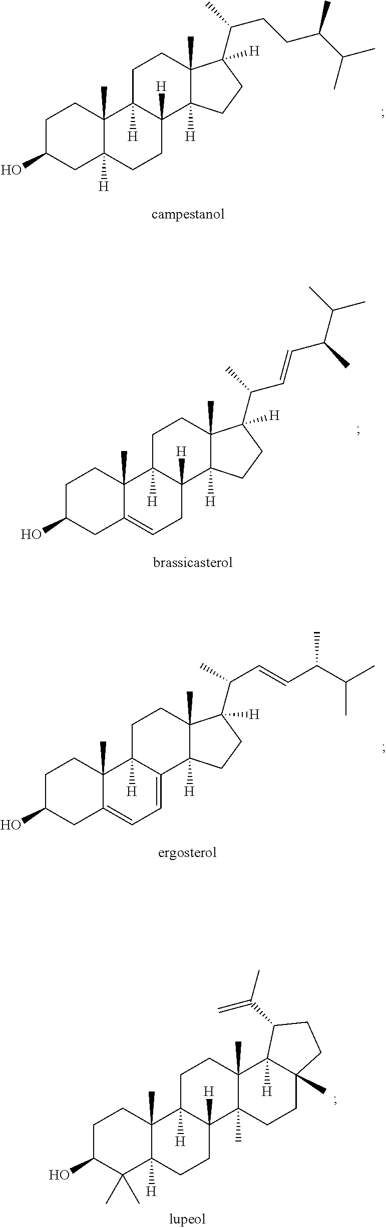

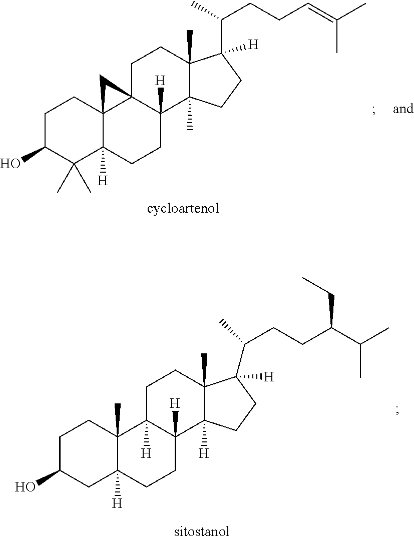

or a pharmaceutically acceptable salt, hydrate, prodrug or optical isomer thereof.

In one embodiment, the compound of formula (I) is cholesterol, sitosterol, campesterol, stigmasterol, campestanol, brassicasterol, ergosterol, lupeol, cycloartenol or sitostanol. In another embodiment, the compound of formula (I) is a pharmaceutically acceptable salt of cholesterol, sitosterol, campesterol, stigmasterol, campestanol, brassicasterol, ergosterol, lupeol, cycloartenol or sitostanol. In another embodiment, the compound of formula (I) is a hydrate of cholesterol, sitosterol, campesterol, stigmasterol, campestanol, brassicasterol, ergosterol, lupeol, cycloartenol or sitostanol.

In the disclosure herein, any reference to formula (I) may be replaced with any of formulas (Ia), (Ib, (Ic), (Id), (Ie), (If), (Ig), (Ih), (Ii), (Ij), (Ik), (Il) or (Im).

### Cholesterol

A particularly preferred compound of formula (II), for example formula (I), is cholesterol. In some embodiments, the one or more compounds of formula (II), for example formula (I), of the composition of the invention is cholesterol and one or more other compounds of formula (II), for example formula (I).

Cholesterol consists of four linked aromatic hydrophobic rings, a small hydrophilic hydroxyl group at the C3 position, and a hydrophobic chain. Due to its very high hydrophobicity, cholesterol is only present within cells as a component of lipid membranes (80-90% of total) or bound to lipid-binding proteins. In cell membranes, it is inserted perpendicular to the membrane plane, with the hydroxyl group at the outer part of the membrane.

The cholesterol of the present invention is any cholesterol that is suitable for use in the methods of the invention. As such, the cholesterol is in a state that can be administered to a subject. The cholesterol may be bound to a compound that is fat-soluble. For example, the cholesterol may be bound to a lipoprotein, for example very-low density lipoprotein (VLDL), low-density lipoprotein (LDL), high-density lipoprotein (HDL), or natural or recombinant apolipoproteins and apolipoprotein mimetics. The cholesterol may be formulated within a liposome formulation. The cholesterol may be formulated with any of the carriers described in the “Carriers of the invention” section.

### Phytosterol

Other particularly preferred compounds of formula (II), for example formula (I), are phytosterols. Phytosterols are analogues of cholesterol that are found in plants. Phytosterols have often been used in subjects to reduce high plasma cholesterol levels, often orally, and thus through impairing gut absorption of cholesterol. However, administered intravenously, it will replace ‘natural’ eukaryotic cholesterol in cell membranes. When referring to one or more compounds of formula (II), for example formula (I), herein, such compounds include one or more phytosterols.

The one or more phytosterols that may be used in the invention is any phytosterol that is suitable for use in the methods of the invention. As such, the one or more phytosterols are in a state that can be administered to a subject. The one or more phytosterols may be bound to any one of the carriers described in the “Carriers of the invention” section.

Phytosterols have several additional advantages compared to cholesterol. For example, phytosterols are more stable than cholesterol, because they are more slowly metabolized. As such, reaching a stable therapeutic dose should be easier than for cholesterol. This also means that less phytosterol and less material that is needed to formulate the phytosterol (e.g. lipids) is required. The present inventors have also shown that up to 30% of cholesterol in a cell membrane may be replaced by phytosterol without toxicity.

### Compositions of the Invention

The present invention provides a composition comprising one or more compounds of formula (II), for example one or more compounds of formula (I). The one or more compounds of formula (II), for example one or more compounds of formula (I), are as described in the sections above. The one or more compounds of formula (II), for example one or more compounds of formula (I), may be a cholesterol or one or more phytosterols.

The present invention also provides a composition comprising two or more compounds of formula (II), for example two or more compounds of formula (I). The two or more compounds of formula (II), for example two or more compounds of formula (I), are as described in the sections above. One of the two or more compounds of formula (II), for example one of the two or more compounds of formula (I), may be a cholesterol, and one or more of the other compounds of formula (II), for example one or more of the other compounds of formula (I), may be one or more phytosterols.

When at least one of the one or two or more compounds of formula (II), for example formula (I), of the composition of the invention is a cholesterol, the concentration ratio of the one or more other compounds of formula (II), for example formula (I), to cholesterol is anywhere from 99:1 to 1:99. For example, the concentration ratio of one or more compounds of formula (II), for example formula (I), to cholesterol may be about 99:1, 95:5, 90:10, 80:20, 75:25, 70:30, 60:40, 50:50, 40:60, 30:70, 25:75, 20:80, 10:90, 5:95 or 1:99. Preferably the concentration ratio of one or more compounds of formula (II), for example formula (I), to cholesterol is between 75:25 to 25:75, for example 70:30 to 30:70, 60:40 to 40:60, 55:45 to 45 to 55, or 50:50.

The composition of the invention may be a pharmaceutical composition comprising a blend of one or more other compounds of formula (II), for example formula (I), and cholesterol. The concentration ratio of the one or more compounds of formula (II), for example formula (I), to cholesterol in the pharmaceutical composition is anywhere from 99:1 to 1:99. For example, the concentration ratio of the one or more compounds of formula (II), for example formula (I), to cholesterol may be 99:1, 95:5, 90:10, 80:20, 75:25, 70:30, 60:40, 50:50, 40:60, 30:70, 25:75, 20:80, 10:90, 5:95 or 1:99. Preferably the concentration ratio is between 75:25 to 25:75, for example 70:30 to 30:70, 60:40 to 40:60, or 55:45 to 45 to 55.

The composition of the invention may be formulated with a carrier, preferably a pharmaceutically acceptable carrier, as defined in the “Carriers of the invention” section.

When (a) the composition of the invention comprises cholesterol and one or more other compounds of formula (II), for example cholesterol and one or more other compounds of formula (I); (b) the composition of the invention further comprises a pharmaceutically acceptable carrier; and (c) the pharmaceutically acceptable carrier comprises cholesterol, the concentration ratio of the one or more other compounds of formula (II), for example formula (I), in the composition to cholesterol in the composition and the pharmaceutically acceptable carrier may be from 75:25 to 25:75, for example 70:30 to 30:70, 60:40 to 40:60, 55:45 to 45 to 55, or about 50:50.

When: (a) the composition comprises one or more compounds of formula (II), for example one or more compounds of formula (I), but not cholesterol;
(b) the composition further comprises a pharmaceutically acceptable carrier; and (c) the pharmaceutically acceptable carrier comprises cholesterol, the concentration ratio of the one or more compounds of formula (II), for example formula (I), that is not cholesterol in the composition, to cholesterol in the pharmaceutically acceptable carrier, is from 75:25 to 25:75, for example 70:30 to 30:70, 60:40 to 40:60, 55:45 to 45 to 55, or about 50:50. In this embodiment, cholesterol is only present in the carrier.

The composition comprising cholesterol and one or more other compounds of formula (II), for example formula (I), can be prepared by measuring the concentration of each compound according to the appropriate ratio and combining them together. The concentration and composition ratio may be measured using standard tools in the art, for example LC-MS (mass spec). The resulting composition may be formulated with a carrier, for example e.g. any of the carriers according the “Carriers of the invention” section.

When formulated as a blend together with of one or more compounds of formula (II), for example formula (I), the cholesterol may be any cholesterol that is suitable for combining with of one or more compounds of formula (II), for example formula (I). For example, the cholesterol may be bound to a lipoprotein, for example very-low density lipoprotein (VLDL), low-density lipoprotein (LDL), high-density lipoprotein (HDL), or natural or recombinant apolipoproteins and apolipoprotein mimetics.

The term “about” or “around” when referring to a value refers to that value but within a reasonable degree of scientific error. Optionally, a value is “about x” if it is within 10%, within 5%, or within 1% of x. Similarly, if the concentration ratio of the one or more compounds of formula (II), for example formula (I), to cholesterol is “about” or “around” 50:50, the amount is within 10%, within 5%, or within 1% of 50:50 i.e. the concentration ratio of the one or more compounds of formula (II), for example formula (I), to cholesterol is between 45:55 to 55:45, 47.5:52.5 to 52.5:47.5, or 49:51 to 51:49.

The concentration ratio as described herein is preferably a mass ratio.

### Carriers of the Invention

The present invention also provides a pharmaceutically acceptable carrier. The carrier is any carrier that can be used to facilitate the administration of the compositions of cholesterol and/or one or more compounds of formula (II), for example formula (I), of the invention to a subject.

The pharmaceutically acceptable carrier may be a nanocarrier. The nanocarrier may be selected from the group consisting of: a micelle, liposomes, nanoparticles, nanoworms and nanorods, or natural or recombinant apolipoproteins and apolipoprotein mimetics. Preferably the carrier is a liposome.

### Liposomes

A preferred carrier of the invention is a liposome. A liposome is known to one of skill in the art as a spherical vesicle comprising at least one phospholipid bilayer. When the cholesterol and/or one or more compounds of formula (II), for example formula (I), of the invention are formulated within a liposome, the cholesterol and/or one or more compounds of formula (II), for example formula (I), are encapsulated within it.

The liposome may be any liposome that is suitable for administering the cholesterol and/or one or more compounds of formula (II), for example formula (I), of the invention. It may be a multilamellar vesicle or unilamellar vesicle, preferably a multilamellar vesicle. The liposome can be prepared by standard methods in the art.

The cholesterol and/or one or more compounds of formula (II), for example formula (I), can loaded into the liposome by standard methods in the art, such as the passive loading techniques (i.e. encapsulation during liposome formulation) or active loading techniques (i.e. encapsulation after liposome formulation).

The liposome may be formulated such that it can be delivered to cells without being cleared by immune cells from the bloodstream. For example, the liposome may be coated with PEG, or synthetic phospholipids may be used. The liposomal formulations of the invention may be formulated according to known methods in the art, for example in Akbarzadeh et al (Akbarzadeh A, Rezaei-Sadabady R, Davaran S, Joo SW, Zarghami N, Hanifehpour Y, Samiei M, Kouhi M, Nejati-Koshki K. Liposome: classification, preparation, and applications. Nanoscale Res Lett. 2013 Feb. 22; 8(1):102. doi: 10.1186/1556-276X-8-102. PMID: 23432972; PMCID: PMC3599573) and Bulbake et al (Bulbake U, Doppalapudi S, Kommineni N, Khan W. Liposomal Formulations in Clinical Use: An Updated Review. Pharmaceutics. 2017 Mar. 27; 9(2):12. doi: 10.3390/pharmaceutics9020012. PMID: 28346375; PMCID: PMC5489929).

The liposomal formula may comprise of an active compound and at least one lipid. The active compound may be a sterol e.g. a cholesterol or a compound with the formula (II), for example formula (I). The lipid may be an amphiphilic molecule that allows the formulation of liposomes such as HSPC, DSPE, DOPC, DSPC, DPPG, DPPC, EPC, POPC and/or SM. Further chemical modifications on the lipid may be present to direct the active compound to the liver or heart, such as sugars or stealth-polymers (e.g. polyethylene glycols, polyoxazolines).

### Subject/Subject in Need Thereof

In any of the methods of the invention disclosed herein, the term “subject” or “subject in need thereof” may be a human. The most preferred subject to which the methods of the invention are applicable are humans.

In any of the methods of the invention disclosed herein the “subject” or “subject in need thereof” may be a non-human animal. For example, methods of the invention disclosed herein may be applied to non-human animals, for example to determine the efficacy of new therapeutics, new therapeutic strategies, new modes of administration of pre-existing therapeutic strategies, or surgical methods. Thus in any of the methods of the invention disclosed herein the subject” or “subject in need thereof” may be a mammal, such as a feline mammal, a canine mammal, a porcine mammal, an equine mammal, a bovine mammal, a rodent mammal, a murine mammal e.g. a mouse or a rat, or a primate mammal e.g. a monkey or chimpanzee.

### Diseases

The compositions of the invention may be used in any method of therapy practised on the human or animal body.

The disease which can be treated by the compositions or methods of the present invention may be any disease associated with myocardial depression. Therefore, the disease may be any disease where myocardial depression is at least one of symptoms. Myocardial depression can be defined as systolic and/or diastolic dysfunction of left and/or right sides of the heart. In practice, myocardial depression, and the severity of myocardial depression, is defined by a variety of experimental tests and markers. For example, it can be identified by biomarkers of ventricular dysfunction such as brain natriuretic peptide (BNP), by echocardiography, or by other techniques that also measure cardiac function such as Doppler blood flow velocimetry. It can also be identified by non-responsiveness to adrenergic agonists such as dobutamine. The compositions and methods of the present invention may treat myocardial depression regardless of the severity. The compositions and methods of the present invention may prevent the progression of myocardial depression to more severe forms of the disease. For example, the compositions and methods of the present invention may prevent the progression of myocardial depression from a mild to a severe form.

The disease associated with myocardial depression may be a disease caused by infectious or non-infectious causes.

Infectious causes include infections caused by a pathogen, such as a bacterium, virus or parasite. Examples of diseases caused by bacterial infections include sepsis, for example bacterial sepsis, such as pneumococcal sepsis. Examples of diseases caused by viral infections include diseases caused by adenoviruses, enteroviruses, influenza and coronaviruses. Examples of diseases caused by parasitic infections include trypanosomiasis.

Non-infectious causes of myocardial depression are those that are not associated with pathogens. Such causes can be related to diseases that a subject may already have and lead to myocardial depression. An example is ischaemic heart disease. Such causes can also be a result of human intervention, such as a reaction to a therapeutic. For example, the non-infectious cause of myocardial depression may be drug toxicity, such as from chemotherapeutics.

The disease which can be treated by the compositions or methods of the present invention may be any disease associated with decreased adrenergic signalling responsiveness, preferably decreased beta-adrenergic signalling responsiveness. The disease can be any disease associated with decreased adrenergic responsiveness to adrenergic agonists, preferably beta-adrenergic agonists. Responsiveness to adrenergic agonists may be defined in terms of stroke volume enhancement. For example, the percentage change in stroke volume can be measured after administration of an adrenergic agonist. A subject with a disease associated with decreased responsiveness to adrenergic agonists will present with a lower percentage change in stroke volume compared to a subject that does not.

The disease associated with decreased adrenergic signalling responsiveness may be a disease caused by infectious or non-infectious causes.

Infectious causes include infections caused by a pathogen, such as a bacterium, virus or parasite. Examples of diseases caused by bacterial infections include sepsis, for example bacterial sepsis, such as pneumococcal sepsis. Examples of diseases caused by viral infections include diseases caused by adenoviruses, enteroviruses, influenza and coronaviruses. Examples of diseases caused by parasitic infections include trypanosomiasis.

Non-infectious causes of myocardial depression are those that are not associated with pathogens. Such causes can be related to diseases that a subject may already have and lead to myocardial depression. An example is ischaemic heart disease. Such causes can also be a result of human intervention, such as a reaction to a therapeutic. For example, the non-infectious cause of myocardial depression may be drug toxicity, such as from chemotherapeutics.

### Methods of Treatment

The compositions of the invention may be used in any method of therapy practised on the human or animal body.

The compositions of the invention can be used to treat myocardial depression and to increase adrenergic signalling responsiveness, preferably beta-adrenergic signalling responsiveness, in a subject. The compositions may be administered intravenously to a subject in need thereof.

The amount of cholesterol and/or one or more compounds of formula (II), for example formula (I), compositions administered to a subject is a “therapeutically effective amount”. A therapeutically effective amount may be a dose sufficient to reduce or eliminate the abnormalities associated with myocardial depression. A therapeutically effective amount may be a dose sufficient to increase adrenergic signalling responsiveness. For example, the amount may be sufficient to restore normal levels of myocardial response to adrenergic agonists such as catecholamine inotropes such as adrenaline or dobutamine.

In an embodiment, the “therapeutically effective amount” may be sufficient to increase the amount of cholesterol in cell membranes. The amount may be sufficient to increase the amount of cholesterol in cardiomyocyte cell membranes. The amount may be sufficient to increase the amount of cholesterol in other cells, such as immune cells, and enhance their functions.

In an embodiment, the “therapeutically effective amount” may be sufficient to restore the amount of sterol e.g. cholesterol in the membrane of cells (e.g. cardiomyocytes) back to normal levels e.g. before myocardial depression onset.

Doses for delivery and administration can be based upon current existing protocols, empirically determined, using animal disease models or optionally in human clinical trials. Initial study doses can be based upon animal studies set forth herein, for a rat, for example.

Doses can vary and depend upon whether the treatment is prophylactic or therapeutic, the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled person will appreciate the factors that may influence the dosage and timing required to provide an amount sufficient for providing a therapeutic or prophylactic benefit.

### Pharmaceutical Compositions

The present invention provides a pharmaceutical composition comprising a blend of cholesterol and one or more other compounds of formula (II), for example formula (I). The ratio of the blend may be determined by the ratios described above.

A pharmaceutical composition according to the present invention may be presented in a form that is ready for immediate use. Alternatively, the composition may be presented in a form that requires some preparation prior to administration.

Pharmaceutical compositions of the invention may be adapted for intravenous administration.

Pharmaceutical compositions adapted for intravenous administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents.

Excipients which may be used for injectable solutions include water, alcohols, polyols, glycerine and vegetable oils, for example. The compositions may be presented in unit-dose or multidose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants.

### EXAMPLES

### Example 1: Materials and Methods

### (a) Reagents and Antibodies

### Chemicals and Reagents:

(i) Cholesterol C₈₆₆₇ Sigma
(ii) Sitosterol SC-204432 Santa Cruz
(iii) Methyl-β-cyclodextrin (MbCD) Sigma 332615

### Assays:

(i) Amplex Red Cholesterol Assay Kit Invitrogen A12216—this assay measures both cholesterol and phytosterol levels.
(ii) Bicinchoninic acid (BCA) assay Thermo Fisher Scientific 23227—protein estimation

### Protease Inhibitors:

(i) Pierce A32955, Phosphatase inhibitors Pierce A32957;
(ii) Clarity TM Western ECL Substrate Bio-Rad Cat. #170-5061;
(iii) Bovine HDL CatN 185-10 Lee Biosolutions

### Antibodies:

(i) pERK Cell Signalling #9101—indicator of adrenergic pathway activation
(ii) Phosphofructokinase (PFK) ProteinTech 55028-I-AP—used as control

### Liposomes

Liposomes containing 34% cholesterol in DPPC (16:0PC; 1,2-dipalmitoyl-sn-glycero-3-phosphocholine) in histidine buffer

### (b) Cell Culture and Cell Treatments

Rat embryonic cardiomyocyte H9C2 cells were cultured in high-glucose DMEM (Gibco 41965-039) supplemented with 10% fetal bovine serum (FBS, Gibco 10270-106) and 100 U/ml of penicillin/streptomycin at 37° C. in a humidified atmosphere of 5% CO₂. Cells were split every 2-3 days.

Two days prior to experimentation, cells were seeded in 6 well plates (Corning N-3516) 3×105 cells/well. Before any treatment. cells were washed twice with prewarmed PBS.

### MbCD Treatment:

Cells were incubated in 5 mM solution MßCD in DMEM for 30 minutes. Before treatment cells were washed twice with prewarmed PBS to remove remaining FBS and prevent contamination of exogenous cholesterol.

### Sterol Treatment:

After MßCD treatment cells were washed twice with prewarmed PBS to remove remaining MßCD solution and then incubated for 4 hours with either (i) complex Cholesterol—MßCD or (ii) Sitosterol—MßCD. These were prepared according to the protocol reported by Klein et al. (Biochemistry 1995; 34:13784). Sterol complexes were diluted ×100 in DMEM to produce final concentrations of 68 microM cholesterol, 68 microM sitosterol and 700 microM MßCD.

### Catecholamine Treatment:

Control, MßCD or MßCD/sterol-treated cells were incubated for 10 minutes with adrenaline or noradrenaline solution in DMEM (final concentration 10⁻⁷M).

c) Liposome Preparation with Sterols

### Instruments/Material

16:0 PC (DPPC) 1,2-dipalmitoyl-sn-glycero-3-phosphocholine* (Avanti® Polar Lipids)
β-Sitosterol* (Santa Cruz Biotechnology #CAS 83-46-5)
Cholesterol* (#8866.2, Carl Roth, #CAS 57-88-5)
Chloroform (HPLC Grade, #7331.1, Carl Roth, #CAS 67-66-3)
Type1 water (Ultrapure water, MilliQ System GE Healthcare)
HEPES buffer pH7 (100 mmol/L stock, diluted to 10 mmol/L final with type 1 water)
Avanti Mini Extruder (Avanti Polar Lipids)
Avanti Filter Support (10 mm) (Merck, Avanti 610014-1EA)
PC membranes 0.1 μm, 19 mm (Merck, Avanti 610005-1EA)
Rotary Evaporator (RV8 V-C, IKA, Germany)
Lyophilization machine (Vacuum freeze dryer, ThermoFisher)
Nitrogen (N₂) gas (Linde Gas, Germany)
   *dissolved in chloroform (25 mg/ml) prior usage.

### Procedure

### 1. Lipid Mixture

Mix the DPPC and cholesterol/sitosterol stock solutions with a desired mass ratio in a round bottom flask (e.g. DPPC:Cholesterol=1:1).

### 2. Lipid Film

Evaporation
   Preheat the rotatory evaporator water bath to circa 40° C.
   Attach the flask, lower down the level to immerse the flask into a water bath, adjust the angle to have the most extensive evaporation surface area.
   Connect the Nitrogen gas to the rotary evaporator to create a protective nitrogen stream on the sample.
   Start rotation (rotation speed: ˜50 rpm for 2 ml of solution, adjust the speed according to the solution volume).
   Evaporate the chloroform to get a dry lipid film.
Lyophilize the samples to remove any solvent residue

### 3. Hydration

Add preheated type 1 water (50° C.) to the dried lipid film to obtain a final nominal lipid concentration of 10 mg/ml.
Stir in 50-60° C. water bath on magnetic stirrer to get homogenous liposome suspension
Optional sonication 10 min to increase the homogeneity of the suspension

### 4. Extrusion

Assembly of the extruder according to manufacturer's protocol
Preheat the extruder on a heat block to 60° C.
Load the liposome suspension into the syringe and start extrusion to obtain a homogenous liposome solution
Extrusion was performed 9 or 11 times (must be an odd numbers to get the final solution in another syringe)

### 5. Storage

The final extruded liposome solution was resuspended in buffer solution (HEPES 10 mmol/L) to obtain the final nominal lipid concentration of 10 mg/ml
It is stored at 4° C.

### d) In Vitro/Ex Vivo Measurements

### Determination of Sterol Concentration in H9C2 Cells:

After incubation MßCD-treated, MßCD/Sterol treated, and untreated (control) cells were washed twice with ice-cold PBS and lysed with phosphate buffer (0.1 M potassium phosphate pH 7.4, 50 mM NaCl, 5 mM cholic acid, 0.1% Triton X-100, protease inhibitors) on ice. Lysates were scraped and collected into Eppendorf tubes, homogenized, vigorously vortexed, sonicated for 15 minutes on ice in the sonication bath, and then centrifuged for 10 minutes at maximum speed and 4° C. Supernatants were transferred into new Eppendorf tubes. Protein and sterol concentrations were measured by BCA and Cholesterol Amplex Red assays, respectively. Results are presented as a ratio of sterol concentration to protein concentration.

Western Blot (WB) Analysis of pERK2 Phosphorylation:

Cells were washed and lysed on plates with RIPA buffer supplemented by protease and phosphatase inhibitors on ice for 30 minutes. Lysates were treated as described above for sterol determination. Protein concentration was determined by BCA assay. Western blot samples were prepared by mixing the lysates with 5× loading buffer (Tris HCl pH 6.8 312.5 mM, SDS 10%, bME 12.5%, glycerol 47.5%, bromophenol blue 0.01%). 2 mcg of protein/sample was separated by electrophoresis on 10% SDS-PAGE and transferred to a PVDF membrane. Membrane was blocked with 5% BSA in TBST for 1 hour at room temperature with gentle agitation and incubated with primary anti-pERK or anti-PFK antibodies overnight at 4° C. Both primary antibodies were diluted 2000 times in 5% BSA in TBST. Membrane was washed 3 times ×10 minutes with TBST and further incubated for 1 h at room temperature with the secondary goat anti-rabbit IgG HRP conjugated antibody diluted with TBST 1:10000 and 1:25000 for PFK and pERK, respectively.

Chemiluminescent detection was performed using the Clarity TM Western ECL Substrate (Bio-Rad). Band intensity was analysed by Image Studio Lite ver5.2. Results are calculated as a ratio of intensities of pERK2 band to PFK band for each sample and normalized as a percentage from the average of samples treated with adrenaline only.

### Plasma Troponin T and BNP Measurement:

Human troponin T was measured by the Royal Free Hospital Chemical Pathology laboratory using a 4th generation high-sensitivity cardiac troponin T electrochemiluminescence immunoassay (Roche Diagnostics, Basel, Switzerland). Rat troponin T was measured using a sandwich ELISA (E-EL-R0151, Elabscience, Beijing, China). BNP levels were measured by competitive ELISA in both rat samples (RAB0386, Sigma-Aldrich) and human samples (EELH0598, Elabscience). Cholesterol measurement in plasma and membrane preparations: Total cholesterol, HDL and LDL cholesterol in plasma samples from patients and rats were measured by the Chemical Pathology Lab, Royal Free Hospital, London.

Measurement of total and free cholesterol in samples from all other experiments including membrane preps from heart and liver tissues was performed in the UCL lab by Amplex Red Cholesterol Assay kit (Invitrogen A12216) according to the manufacturer's protocol.

### (e) In Vivo Model

### Long-Term Fluid-Resuscitated Rat Model Of Faecal Peritonitis

Experiments were performed under Home Office and local Ethics Committee approval in accordance with the Animals (Scientific Procedures) Act 1986. During all studies animals had access to food and water ad libitum. A 12-hour light-dark cycle was maintained. Any animal showing distress (using an in-house validated score) was sacrificed.

Study I. Changes in Plasma, Heart and Liver Cholesterol Levels with Sepsis

Male Wistar rats (350±25 g) were instrumented under isoflurane anaesthesia maintained via a face mask. Internal jugular venous and carotid arterial catheters (external diameter 0.96 mm; Biocorp Ltd, Huntingdale, NSW, Australia) were inserted and tunneled subcutaneously to the nape of the neck. Catheters were mounted onto a swivel/tether system that enabled unimpaired movement around the cage after recovery from anaesthesia.

Sepsis was induced by an i.p. injection of 4 μl/g body weight human faecal slurry diluted in n-saline. Fluid resuscitation (50:50 mix of 5% glucose/Hartmann's solutions; 10 ml/kg/h) was commenced at 2 h after sepsis induction. Sham operated animals were treated identically except for slurry injection.

At 6 h, transthoracic echocardiography was performed using a 14 MHz probe scanning at 0-2 cm depth (Vivid 7 Dimension, GE Healthcare, Bedford, UK). An echo-measured heart rate cut-off of 460 bpm was used to classify animals into predicted survivors (SR) or non-survivors (NSR). This has been previously validated in this model to predict survival with approximately 90% accuracy. Importantly, in this model, non-survivors usually die between 20-48 h while survivors are showing clear signs of clinical improvement at 72 h (study end).

At 6 h, 24 h or 72 h (termination of study) after induction of sepsis, rats were sacrificed. Heart and liver tissue samples were collected and snap frozen in liquid nitrogen. Blood from the descending aorta was collected into tubes containing EDTA and centrifuged at 6500 g for 15 minutes. Plasma was separated, aliquoted and snap-frozen in liquid nitrogen. All samples were stored at −80° C. for later analysis.

All tissue samples were stored at −80° C., subsequent manipulations of tissue samples were performed either on dry ice or in liquid nitrogen.

Frozen tissue was pulverized in liquid nitrogen; 50 mg was homogenized in 1 ml ice-cold Membrane buffer (10 mM Tris HCl 50 mM NaCl, protease inhibitors) in a 7 ml Dounce homogenizer. Homogenates were transferred into Eppendorf tubes, centrifuged twice at 4° C. for 10 minutes at 1000 g. On both occasions, pellet was discarded and supernatant transferred into new tubes. 500 μl supernatant was transferred into a centrifuge tube (Beckman Counter tube Ultra Clear 9/16×3½ in (14×89 mm)), 50 mM Tris HCl pH 7.4 was added to the rim. Samples were then centrifuged for 1.5 hours at 4° C., 30000 rpm Rotor SW41. After centrifugation, the supernatant was discarded and the pellet dissolved in 300 microl buffer (10 mM TrisHCl pH 7.4, 50 mM NaCl, 1 mM EDTA, 1 mM EGTA, 0.1% Triton×100). This was then vortexed vigorously and passed 10-20× through a 27 G needle syringe, centrifuged at 4° C. for 10 min at 14000 rpm. The protein concentration in the membrane preparation was determined by BCA assay and cholesterol by Amplex Red (Invitrogen).

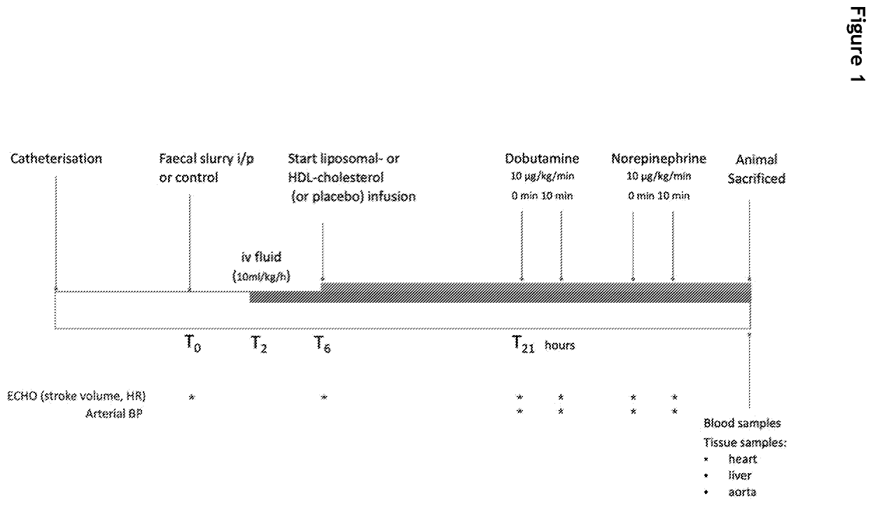

### Study 2: Catecholamine Responsiveness Study (FIG. 1)

II. Sepsis was induced in awake, instrumented male Wistar rats (316±23.5 g) as described above but using 7 microL of faecal slurry/g body weight. Rats were allocated into sham-operated (control), sepsis and sepsis+cholesterol groups.

At 6 h post-induction of sepsis, the sepsis+cholesterol group received an i.v. infusion of a cholesterol-loaded formulation, either liposomal- or bovine HDL (10 mg cholesterol/300 g bw), which was continued over 15 hours.

At 21 h, catecholamine responsiveness was tested by consecutively administering, under isoflurane anaesthesia, the beta-adrenergic agonist, dobutamine (10 μg/kg/min for 10 min) and then the predominant alpha-adrenergic agonist, norepinephrine (0.5 μg/kg/min for 10 min) with a 30 min washout period in between.

Blood pressure (measured continuously from an indwelling carotid line), heart rate and stroke volume (measured by echocardiography) were measured before and at 10 minutes after each drug was infused.

### f) Septic Patients

Patients in septic shock requiring vasopressor treatment were enrolled from the intensive care unit of University College Hospital. Ethics Committee permission was obtained and informed consent sought from the patient or their next-of-kin if they lacked competency. Retrospective consent was obtained from surviving patients.

Twenty ml blood was taken from indwelling arterial lines on Days 0-3 of their ICU admission. The blood was promptly centrifuged and the plasma separated off into 2 ml aliquots and promptly frozen at −20° C. Samples were subsequently transferred to a −80° C. freezer for longer-term storage prior to batch analysis.

### g) Statistical Analysis

Results are presented as the mean±standard error. Statistical analysis was performed by one-way ANOVA or by Student's t-test (GraphPad Software, San Diego, CA, USA). Significance was defined as P<0.05.

### Example 2: Cholesterol Falls in Septic Patients (Plasma) and Rats (Plasma and Heart/Liver Cell Membrane)

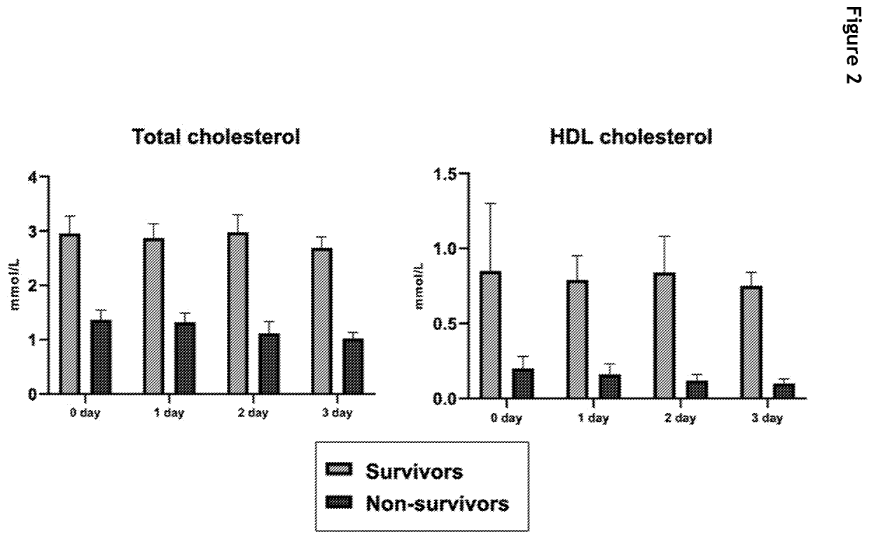

### (i) Patient Study (FIG. 2)

21 patients in septic shock (12 eventual survivors, 9 non-survivors) were studied.

Plasma levels of total and HDL-cholesterol were significantly lower (p<0.05) in septic patients compared to healthy controls (normal range in adults total cholesterol 4-5 mmol/l, HDL cholesterol 0.93-1.44 mmol/l). Levels were significantly lower in eventual non-survivors (p<0.05). Levels remained stable over the 4-day observation period.

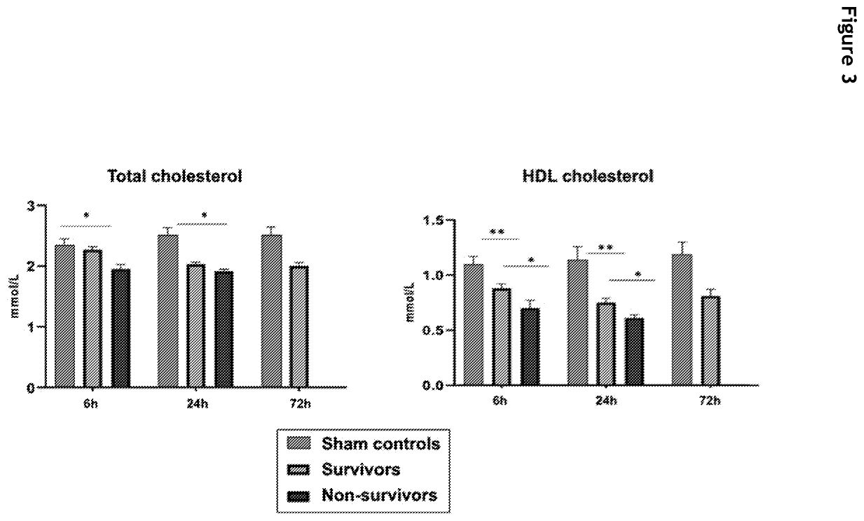

### (ii) In Vivo Rat Study (FIG. 3)

Septic rats also showed a rapid fall in plasma cholesterol levels with sepsis (FIG. 3). This was seen as early as 6 h after sepsis onset. Levels were significantly decreased in both predicted survivors and non-survivors. Importantly, like in human sepsis the drop of cholesterol in septic rats was significantly stronger in non-survivors over the whole observation period.

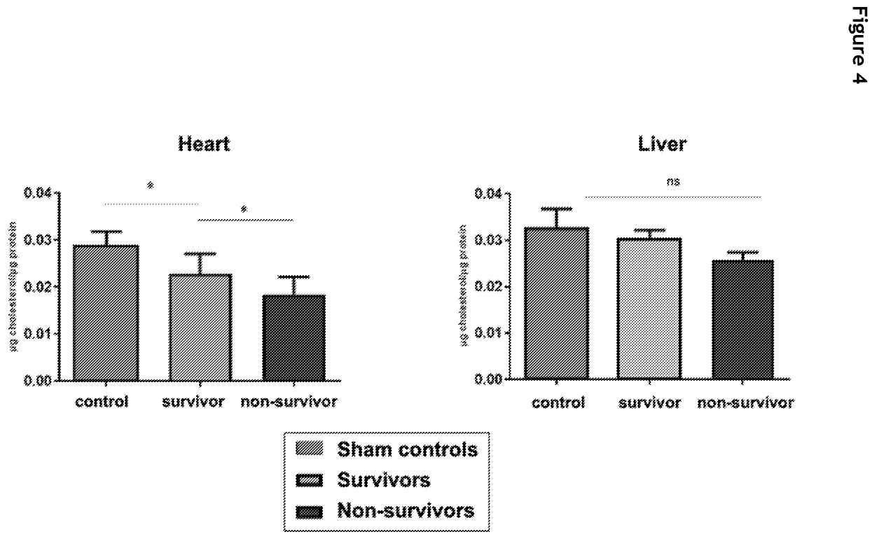

Cholesterol concentration in the membrane preparations from hearts and livers from naïve and septic animals are shown in FIG. 4. Membrane cholesterol fell significantly in the cardiomyocyte membranes obtained from septic rats at 24 h, more so in non-survivors. Cholesterol in liver cell membranes showed the same tendency in septic rats, albeit non-significant

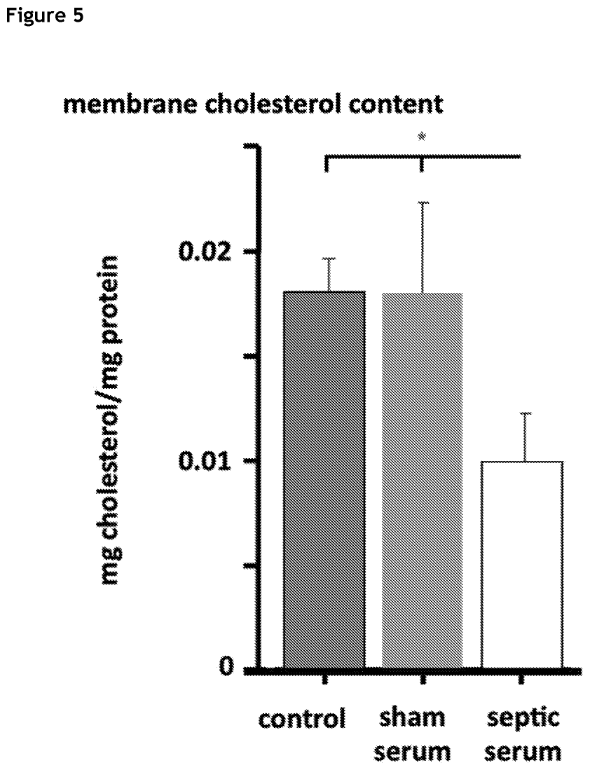

The fall in cardiomyocyte membrane cholesterol was recapitulated by incubating H9C2 cardiomyocytes for 2 h with pooled serum taken from either healthy rats or septic rats at 24 h (FIG. 5).

### Example 3: Myocardial Injury and Dysfunction and Cardiovascular Alterations in Septic Patients and Rats

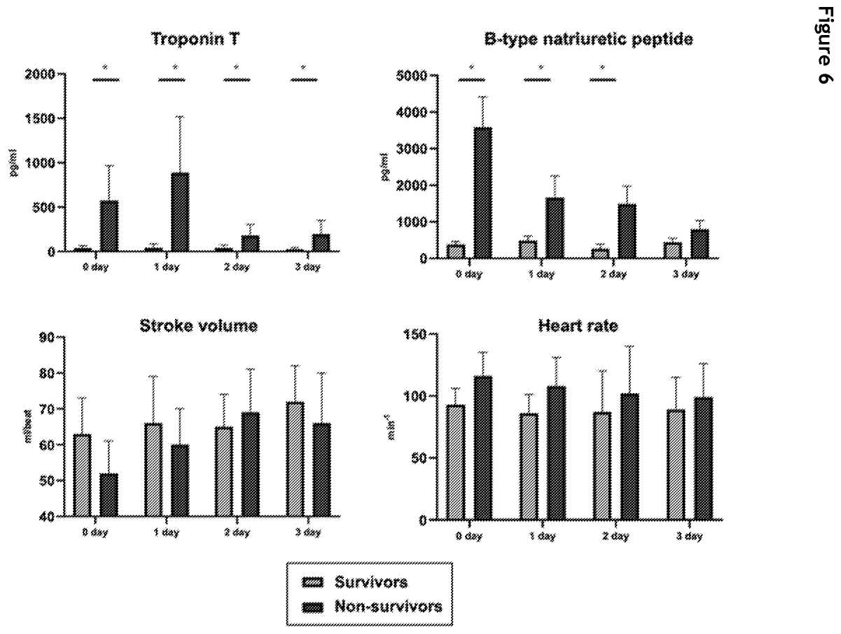

In septic shock patients, biomarkers of myocardial injury (Troponin T) and ventricular dysfunction (brain natriuretic peptide, BNP) were increased in survivors and markedly elevated in eventual non-survivors. This was apparent from the day of ICU admission onwards (FIG. 6).

Stroke volume was lower and heart rate higher in eventual non-survivors of septic shock, albeit non-significant.

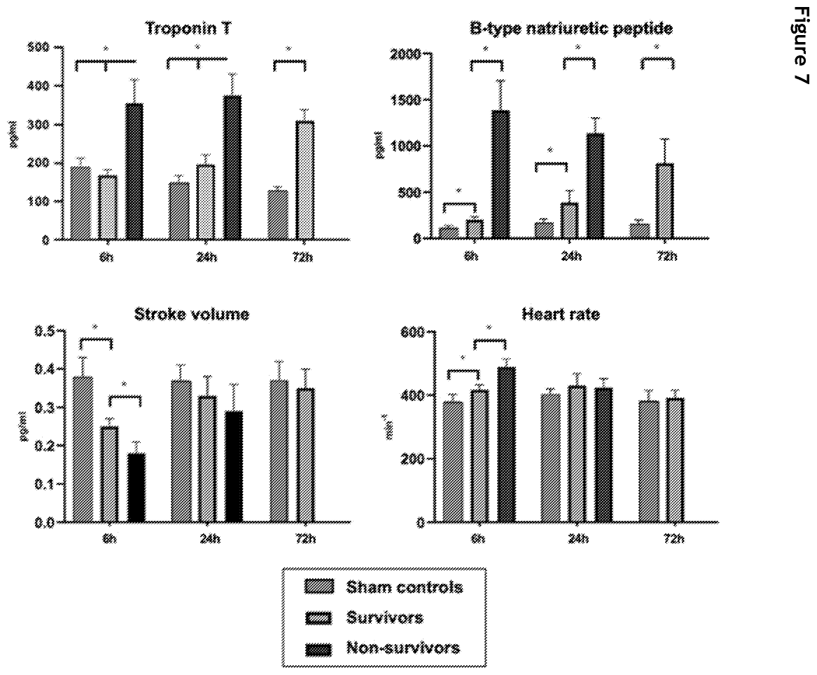

The same pattern was seen in the septic rats (FIG. 7) with eventual non-survivors showing greater pertubations.

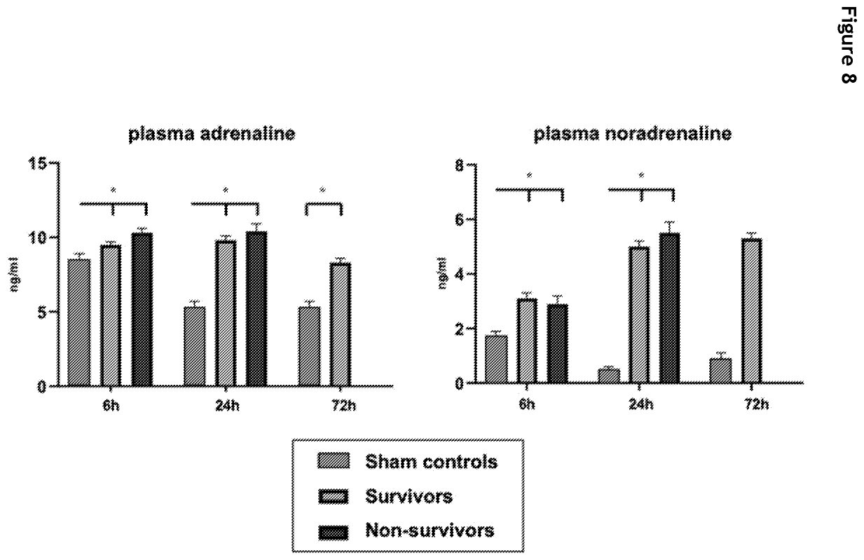

As expected from a stressed state, the septic rats generated an increase in plasma catecholamine levels which was significantly greater than controls (FIG. 8). However, no differences were seen between survivors and non-survivors at any of the timepoints analysed.

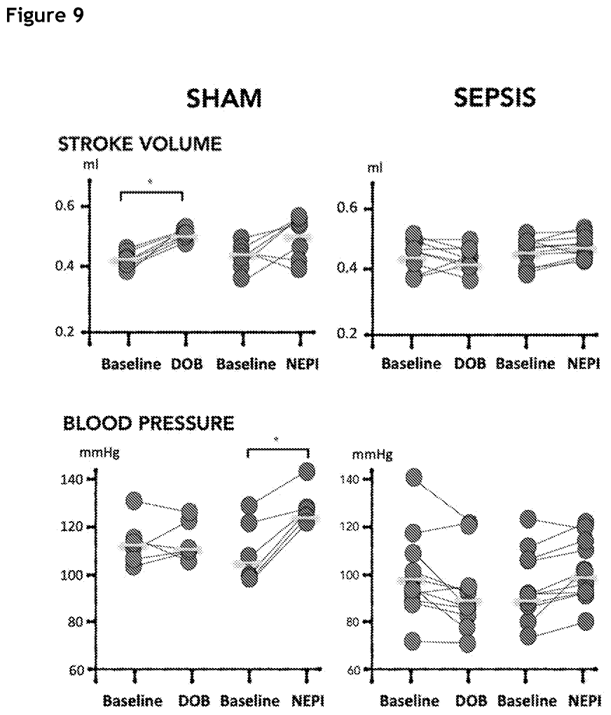

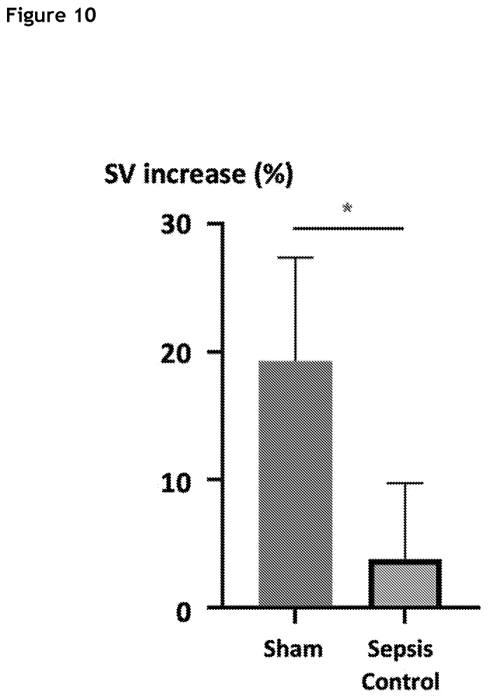

Notwithstanding this increase in plasma catecholamine levels, the animals were non-responsive in terms of stroke volume response to the predominant beta-adrenergic inotropic agonist, dobutamine, nor in blood pressure response to the predominant alpha1-adrenergic vasopressor agonist, noradrenaline measured 10 minutes after commencement of infusion (FIG. 9). The sham animals, by contrast, showed significant rises in stroke volume with dobutamine and blood pressure with noradrenaline. The percentage change in stroke volume with dobutamine is shown in FIG. 10.

As well as recapitulating other physiological and biochemical features of human sepsis, this animal model demonstrates catecholamine hyporeactivity, a hallmark of human sepsis.

### Example 4: Interventions with Cholesterol

### (i) Restoration of Membrane Cholesterol Level Rescues the Myocardial Response to Dobutamine.

Septic animals were treated ether with bovine HDL-cholesterol or with a liposomal cholesterol formulation commencing at 6 h after sepsis initiation and continuing for 15 hours followed by consecutive administration of dobutamine and noradrenaline (as per FIG. 1). At termination of these studies the animals were sacrificed and the heart removed for analysis.

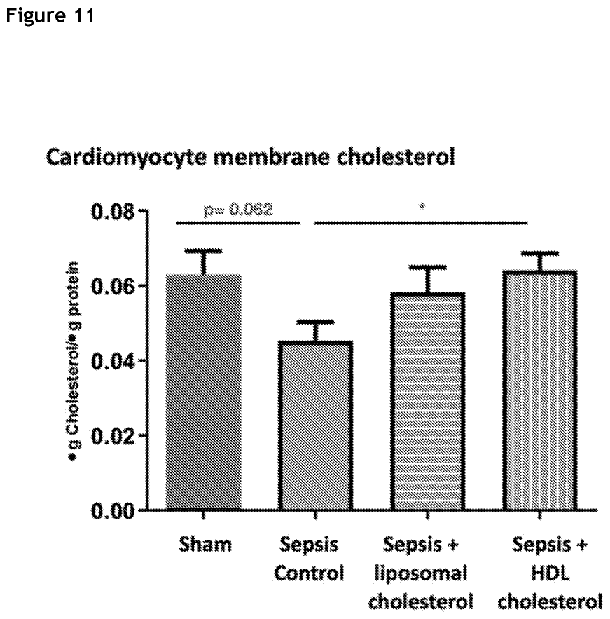

Treatment with either HDL-cholesterol or cholesterol-containing liposomes restored membrane cholesterol levels in the septic hearts to the normal range (FIG. 11).

### (ii) Elevation of Plasma Cholesterol Level

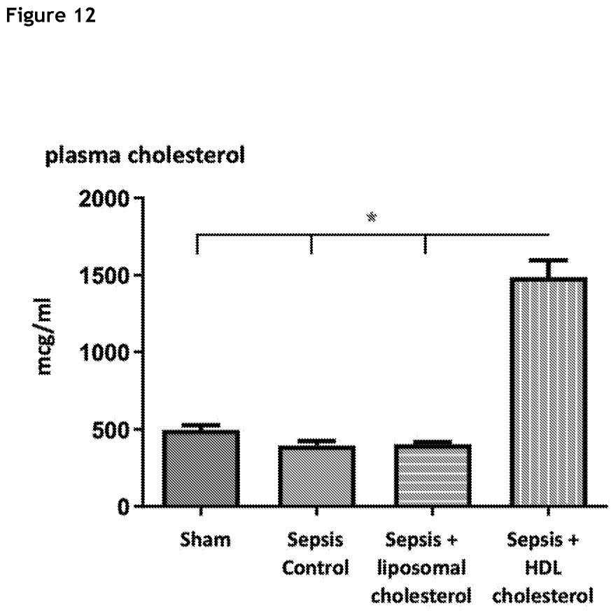

Despite equivalent doses of cholesterol given intravenously over 15 hours either within liposomes or bound to HDL, and equivalent elevations in cardiomyocyte membrane cholesterol content (FIG. 11), the plasma levels of cholesterol were supranormal (3× increase) with bovine HDL-cholesterol whereas little change was seen with liposomal cholesterol (FIG. 12). Reasons are still to be determined but this may relate to different pharmacokinetics with rapid cellular uptake of liposomes.

(iii) Pressor Response to Noradrenaline.

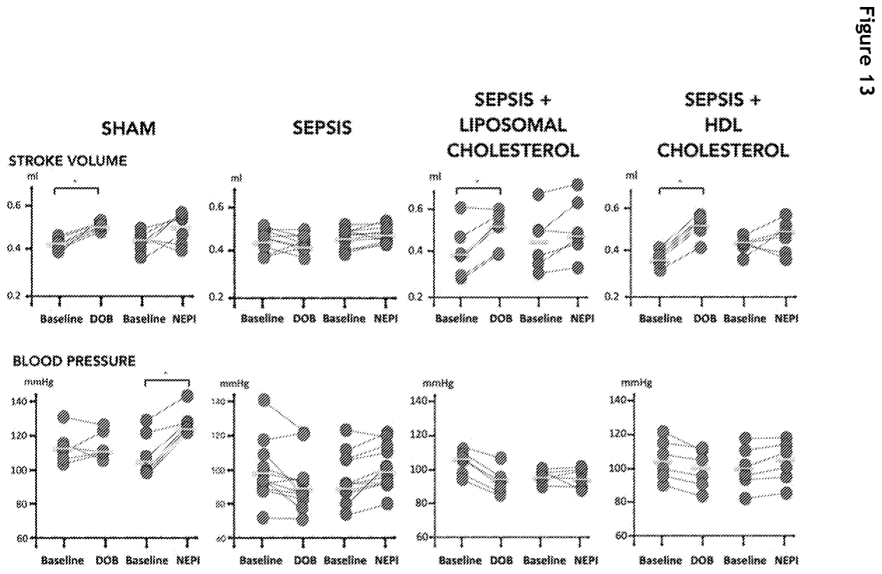

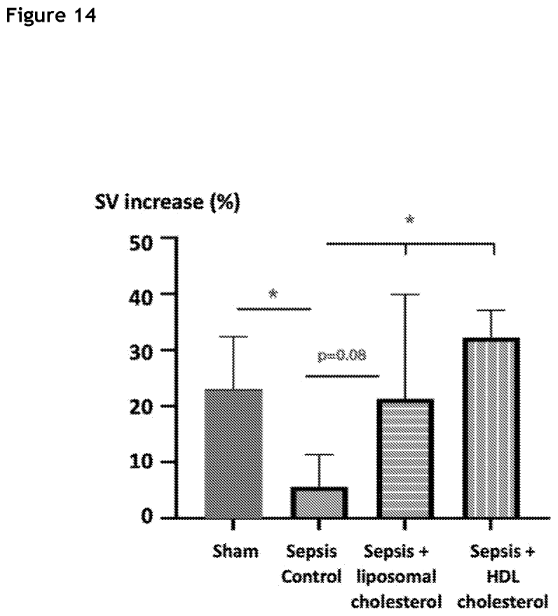

Rats treated with either cholesterol-containing formulation demonstrated a concurrent pronounced improvement in the stroke volume response to dobutamine, indicating a restoration of inotropic effect and reversal of catecholamine hyporeactivity (FIG. 13). The percentage change in stroke volume with dobutamine is shown in FIG. 14. No improvement was seen in the suppressed blood pressure response to norepinephrine in the septic animals, indicating the mode of action of cholesterol therapy is primarily directed towards beta-adrenergic rather than alpha-adrenergic receptors.

### Example 5: Interventions with Phytosterol

(i) Replenishment of Depleted Cellular Membrane Sterol Level with Sitosterol

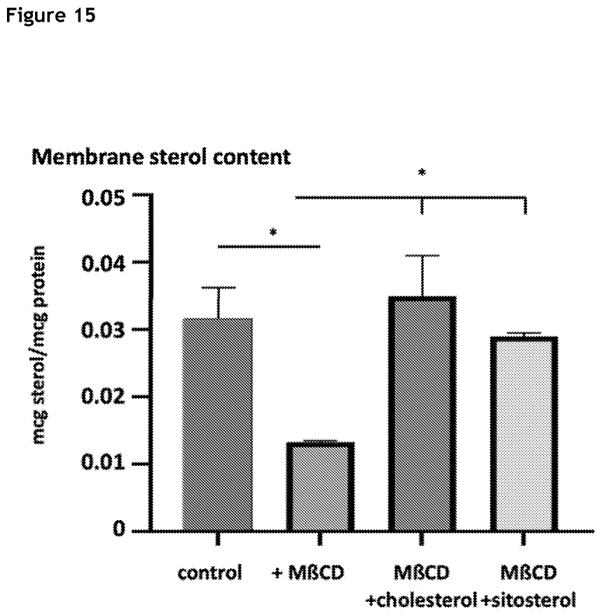

The rat H9C2 cardiomyocyte cells were treated with methyl-β-cyclodextrin (MßCD) to deplete membrane cholesterol content and then incubated with either MßCD-cholesterol or MßCD-sitosterol. Sterol content was restored following treatment with both sterols (FIG. 15).

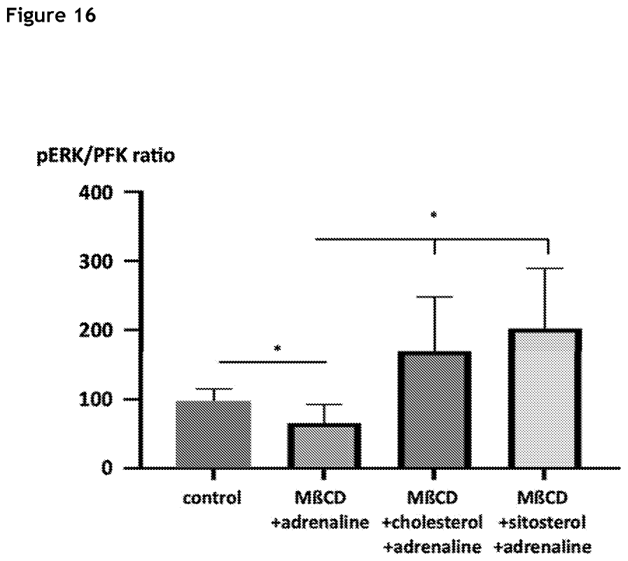

Adrenergic signalling was evaluated in the H9C2 cell line by the increase of ERK2 phosphorylation after adrenaline treatment. Depletion of membrane cholesterol by treatment with MßCD decreased ERK2 phosphorylation but this was enhanced to 2× normal by incubation with either cholesterol or sitosterol (FIG. 16).

### Example 6: Interventions with Liposomes Carrying Cholesterol/Phytosterol

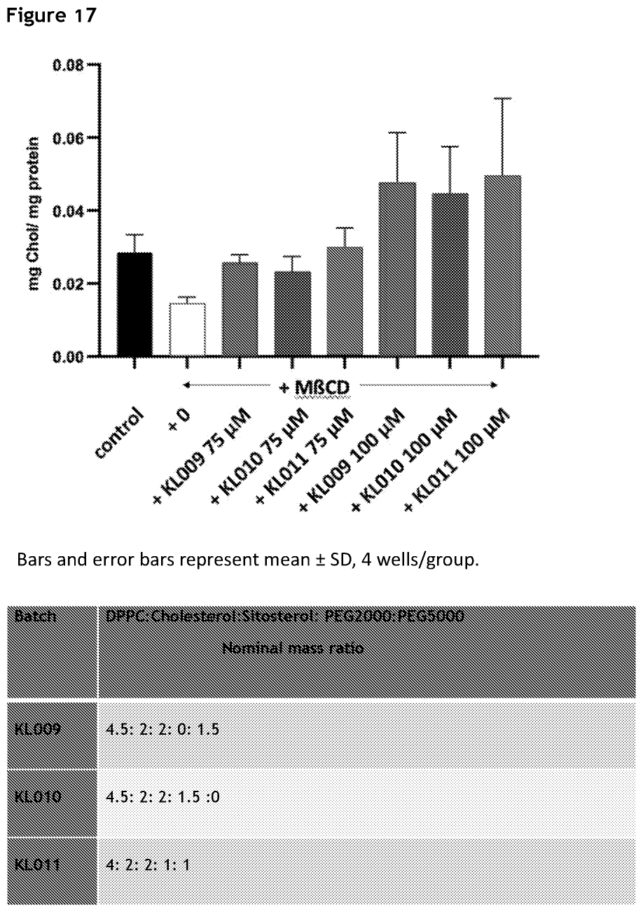

The rat H9C2 cardiomyocyte cells were treated with methyl-ß-cyclodextrin (MßCD) to deplete membrane cholesterol content and then incubated with pegylated liposomal formulations containing the equal amount of cholesterol and ß-sitosterol (the precise description of the formulations is shown in the table below the graph in FIG. 17). Cells were treated with either 75 microM or 100 microM sterols for 1 hour. Cellular sterol content was restored in a dose-dependent fashion. (FIG. 17)

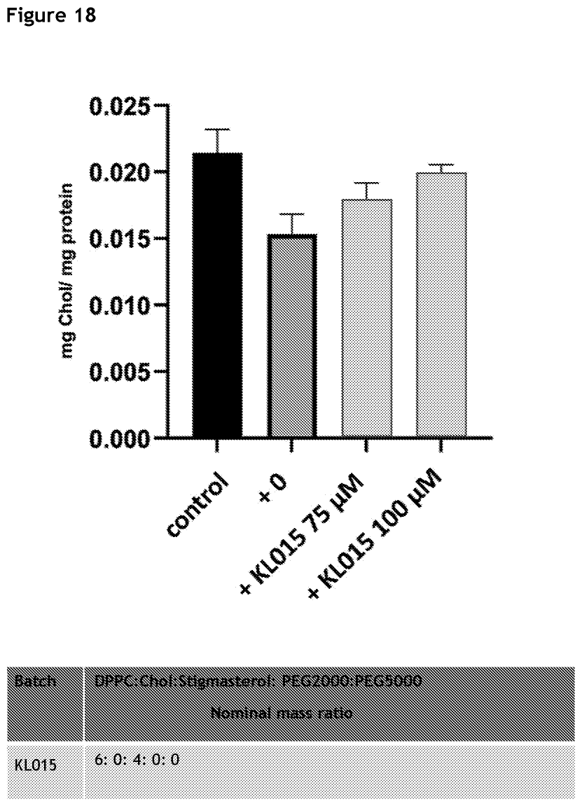

The rat H9C2 cardiomyocyte cells were treated with methyl-ß-cyclodextrin (MßCD) to deplete membrane cholesterol content and then incubated with liposomal formulations containing stigmasterol (the precise description of formulations is shown in the table below the graph in FIG. 18). Cells were treated with either 75 microM or 100 microM sterols for 1 hour. Cellular sterol content was restored in a dose-dependent fashion. (FIG. 18)

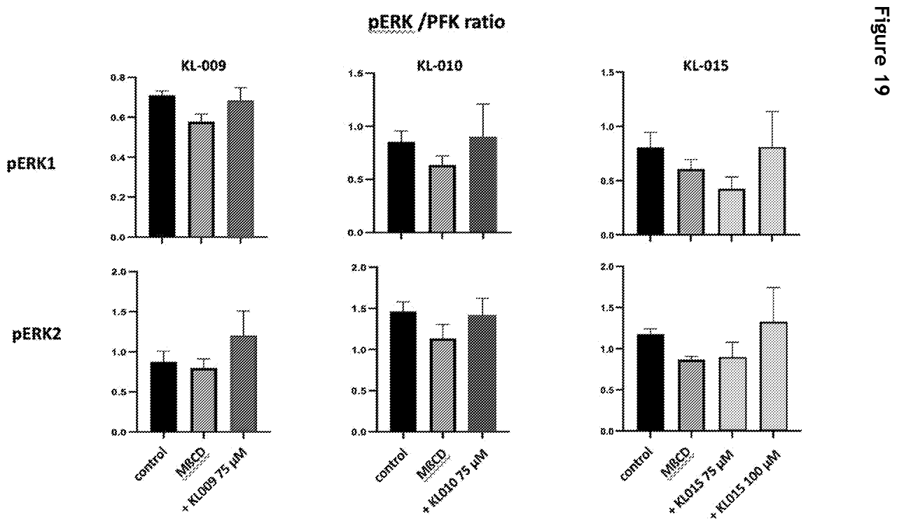

Adrenergic signalling was evaluated in the H9C2 cardiomyocytes by the increase of ERK1 and ERK2 phosphorylation after dobutamine treatment. Depletion of membrane cholesterol by treatment with MßCD decreased phosphorylation of both ERK1 and ERK2 but this was enhanced to normal levels by incubation with pegylated liposomal formulations containing equal amounts of cholesterol and ß-sitosterol or with a non-pegylated liposomal formulation containing stigmasterol (FIG. 19).

### Embodiments of the Invention

1. A method for the treatment of a disease associated with myocardial depression comprising administering to a subject in need thereof a therapeutically effective amount of a composition comprising one or more compounds of formula (II) or a pharmaceutically acceptable salt, hydrate, prodrug or stereoisomer thereof,

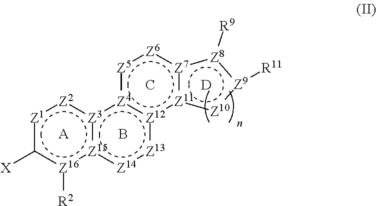

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R is selected from hydrogen, fluorine and optionally substituted C₁₋₂₀ alkyl, or may be absent, or R⁴ and R are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

2. A method of increasing adrenergic signalling responsiveness in a subject in need thereof, comprising administering to the cell, tissue or subject a therapeutically effective amount of a composition comprising one or more compounds of formula (II) or a pharmaceutically acceptable salt, hydrate, prodrug or stereoisomer thereof,

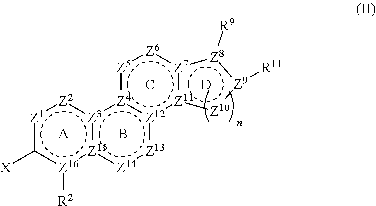

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

3. The method of embodiment 1 or 2, wherein the one or more compounds of formula (II) are one or more compounds of formula (I),

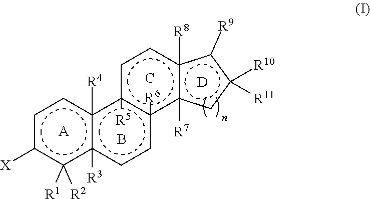

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

4. The method according to embodiment 1 or 2, wherein the one or more compounds of formula (II) is cholesterol and/or one or more phytosterols.

5. The method according to any one of embodiments 1, 2 or 4, wherein the composition comprises two or more compounds of formula (II).

6. The method according to embodiment 5, wherein one of the two or more compounds of formula (II) is cholesterol.

7. The method according to embodiment 5 or 6, wherein one or more compounds of the two or more compounds of formula (II) is one or more phytosterols.

8. The method according to embodiment 3, wherein the one or more compounds of formula (I) is cholesterol and/or one or more phytosterols.

9. The method according to embodiment 3 or 8, wherein the composition comprises two or more compounds of formula (I).

10. The method according to embodiment 9, wherein one of the two or more compounds of formula (I) is cholesterol.

11. The method according to embodiment 9 or 10, wherein one or more compounds of the two or more compounds of formula (I) is one or more phytosterols.

12. The method according to any one of embodiments 1 to 11, wherein the subject is suffering from a disease caused by an infection, optionally wherein the infection is a bacterial infection, a viral infection or a parasitic infection.

13. The method according to any one of embodiments 1 to 12, wherein the disease is sepsis.

14. The method according to any one of embodiments 1 to 11, wherein the subject is suffering from a non-infectious disease, optionally wherein the disease is ischaemic heart disease or drug toxicity.

15. The method according to any one of the preceding embodiments, wherein the composition is administered intravenously to the subject.

16. The method according to any one of the preceding embodiments, wherein the composition further comprises a pharmaceutically acceptable carrier.

17. The method according to embodiment 16, wherein the carrier is a nanocarrier.

18. The method according to embodiment 16 or 17, wherein the carrier is a liposome, micelle, nanoparticle, nanoworm or nanorod.

19. The method according to any one of embodiments 1, 2, 4 to 7 or 12 to 18, wherein the composition comprises cholesterol and one or more other compounds of formula (II), and the concentration ratio of the one or more other compounds of formula (II) to cholesterol is from 75:25 to 25:75.

20. The method according to any one of embodiments 1, 2, 4 to 7 or 12 to 19, wherein:
(a) the composition comprises cholesterol and one or more other compounds of formula (II);
(b) the composition further comprises a pharmaceutically acceptable carrier; and
(c) the pharmaceutically acceptable carrier comprises cholesterol, and
the concentration ratio of the one or more other compounds of formula (II) in the composition to cholesterol in the composition and the pharmaceutically acceptable carrier is from 75:25 to 25:75.

21. The method according to any one of embodiments 1, 2, 4 to 7 or 12 to 20, wherein:
(a) the composition comprises one or more compounds of formula (II) but not cholesterol;
(b) the composition further comprises a pharmaceutically acceptable carrier; and
(c) the pharmaceutically acceptable carrier comprises cholesterol, and
the concentration ratio of the one or more compounds of formula (II) that is not cholesterol in the composition, to cholesterol in the pharmaceutically acceptable carrier, is from 75:25 to 25:75.

22. The method according to any one of embodiments 19 to 21, wherein the concentration ratio of the one or more other compounds of formula (II) to cholesterol, the concentration ratio of the one or more other compounds of formula (II) to cholesterol in the composition and the carrier, or
the concentration ratio of the one or more compounds of formula (II) that is not cholesterol in the composition to cholesterol in the carrier is about 50:50.

23. The method according to any one of embodiments 1, 2, 4 to 7 or 12 to 22, wherein when at least one of the one or more compounds with formula (II) or two or more compounds of formula (II) is a phytosterol, the phytosterol is selected from the group consisting of: sitosterol, campesterol, stigmasterol, campestanol, brassicasterol, ergosterol, lupeol, cycloartenol, and sitostanol.

24. The method according to any one of embodiments 3, or 8 to 18, wherein the composition comprises cholesterol and one or more other compounds of formula (I), and the concentration ratio of the one or more other compounds of formula (I) to cholesterol is from 75:25 to 25:75.

25. The method according to any one of embodiments 3, 8 to 18, or 24, wherein:
(a) the composition comprises cholesterol and one or more other compounds of formula (I);
(b) the composition further comprises a pharmaceutically acceptable carrier; and
(c) the pharmaceutically acceptable carrier comprises cholesterol, and
the concentration ratio of the one or more other compounds of formula (I) in the composition to cholesterol in the composition and the pharmaceutically acceptable carrier is from 75:25 to 25:75.

26. The method according to any one of embodiments 3, 8 to 18, or 24 to 25, wherein:
(a) the composition comprises one or more compounds of formula (I) but not cholesterol;
(b) the composition further comprises a pharmaceutically acceptable carrier; and
(c) the pharmaceutically acceptable carrier comprises cholesterol, and
the concentration ratio of the one or more compounds of formula (I) that is not cholesterol in the composition, to cholesterol in the pharmaceutically acceptable carrier, is from 75:25 to 25:75.

27. The method according to any one of embodiments 24 to 26, wherein the concentration ratio of the one or more other compounds of formula (I) to cholesterol, the concentration ratio of the one or more other compounds of formula (I) to cholesterol in the composition and the carrier, or the concentration ratio of the one or more compounds of formula (I) that is not cholesterol in the composition to cholesterol in the carrier is about 50:50.

28. The method according to any one of embodiments 3, 8 to 18, or 24 to 27, wherein when at least one of the one or more compounds with formula (I) or two or more compounds of formula (II) or (I) is a phytosterol, the phytosterol is selected from the group consisting of: sitosterol, campesterol, stigmasterol, campestanol, brassicasterol, ergosterol, lupeol, cycloartenol, and sitostanol.

29. A pharmaceutical composition comprising: (i) cholesterol and/or (ii) one or more other compounds with formula (II), or a pharmaceutically acceptable salt, hydrate, prodrug or stereoisomer thereof,

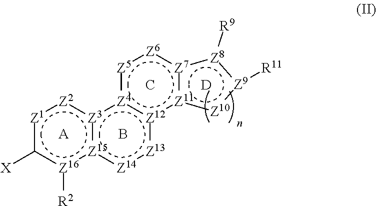

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

30. The composition of embodiment 29, wherein the one or more compounds of formula (II) are one or more compounds of formula (I),

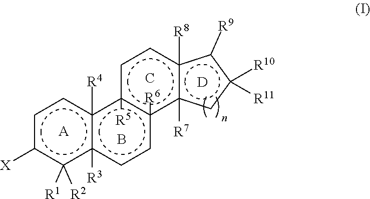

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

31. The composition according to embodiment 29, wherein at least one of the one or more other compounds with formula (II) is a phytosterol.

32. The composition according to embodiment 30, wherein at least one of the one or more other compounds with formula (I) is a phytosterol.

33. The composition according to any one of embodiments 29 to 32, wherein the composition further comprises a pharmaceutically acceptable carrier.

34. The composition according to embodiment 33, wherein the carrier is a nanocarrier.

35. The composition according to embodiment 33 or 34, wherein the carrier is a liposome, micelle, nanoparticle, nanoworm or nanorod.

36. The composition according to any one of embodiments 29, 31, or 33 to 35, wherein the composition comprises cholesterol and one or more other compounds of formula (II), and the concentration ratio of the one or more other compounds of formula (II) to cholesterol is from 75:25 to 25:75.

37. The composition according to any one of embodiments 29, 31, or 33 to 36, wherein:
(a) the composition comprises cholesterol and one or more other compounds of formula (II);
(b) the composition further comprises a pharmaceutically acceptable carrier; and
(c) the pharmaceutically acceptable carrier comprises cholesterol, and
the concentration ratio of the one or more other compounds of formula (II) in the composition to cholesterol in the composition and the pharmaceutically acceptable carrier is from 75:25 to 25:75.

38. The composition according to any one of embodiments 29, 31 or 33 to 36, wherein:
(a) the composition comprises one or more compounds of formula (II) but not cholesterol;
(b) the composition further comprises a pharmaceutically acceptable carrier; and
(c) the pharmaceutically acceptable carrier comprises cholesterol, and
the concentration ratio of the one or more compounds of formula (II) that is not cholesterol in the composition, to cholesterol in the pharmaceutically acceptable carrier, is from 75:25 to 25:75.

39. The composition according to any one of embodiments 36 to 38, wherein the concentration ratio of the one or more other compounds of formula (II) to cholesterol, the concentration ratio of the one or more other compounds of formula (II) to cholesterol in the composition and the carrier, or the concentration ratio of the one or more compounds of formula (II) that is not cholesterol in the composition to cholesterol in the carrier is about 50:50.

40. The composition according to any one of embodiments 29, 31, or 33 to 39, wherein when at least one of the one or more other compounds with formula (II) is a phytosterol, the phytosterol is selected from the group consisting of: sitosterol, campesterol, stigmasterol, campestanol, brassicasterol, ergosterol, lupeol, cycloartenol, and sitostanol.

41. The composition according to any one of embodiments 30, or 32 to 35, wherein the composition comprises cholesterol and one or more other compounds of formula (I), and the concentration ratio of the one or more other compounds of formula (I) to cholesterol is from 75:25 to 25:75.

42. The composition according to any one of embodiments 30, 32 to 35, or 41, wherein:
(a) the composition comprises cholesterol and one or more other compounds of formula (I);
(b) the composition further comprises a pharmaceutically acceptable carrier; and
(c) the pharmaceutically acceptable carrier comprises cholesterol, and
the concentration ratio of the one or more other compounds of formula (I) in the composition to cholesterol in the composition and the pharmaceutically acceptable carrier is from 75:25 to 25:75.

43. The composition according to any one of embodiments 30, 32 to 35, 41 to 42, wherein:
(a) the composition comprises one or more compounds of formula (I) but not cholesterol;
(b) the composition further comprises a pharmaceutically acceptable carrier; and
(c) the pharmaceutically acceptable carrier comprises cholesterol, and
the concentration ratio of the one or more compounds of formula (I) that is not cholesterol in the composition, to cholesterol in the pharmaceutically acceptable carrier, is from 75:25 to 25:75.

44. The composition according to any one of embodiments 41 to 43, wherein the concentration ratio of the one or more other compounds of formula (I) to cholesterol, the concentration ratio of the one or more other compounds of formula (I) to cholesterol in the composition and the carrier, or
the concentration ratio of the one or more compounds of formula (I) that is not cholesterol in the composition to cholesterol in the carrier is about 50:50.

45. The composition according to any one of embodiments 30, 32 to 35, 41 to 45, wherein when at least one of the one or more other compounds with formula (I) is a phytosterol, the phytosterol is selected from the group consisting of: sitosterol, campesterol, stigmasterol, campestanol, brassicasterol, ergosterol, lupeol, cycloartenol, and sitostanol.

46. A composition comprising one or more compounds with formula (II) or a pharmaceutically acceptable salt, hydrate, prodrug or stereoisomer thereof for use in a method of treatment of a disease associated with myocardial depression, the method comprising administering to a subject in need thereof a therapeutically effective amount of the composition,

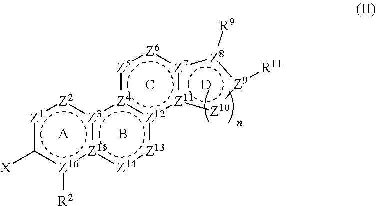

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁷ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

47. A composition comprising one or more compounds with formula (II) or a pharmaceutically acceptable salt, hydrate, prodrug or stereoisomer thereof for use in a method of increasing adrenergic signalling responsiveness in a subject in need thereof, comprising administering to the subject a therapeutically effective amount of the composition,

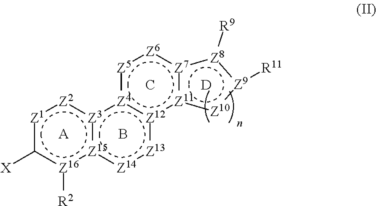

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

48. The composition of embodiment 46 or 47, wherein the one or more compounds with formula (II) are one or more compounds with formula (I),

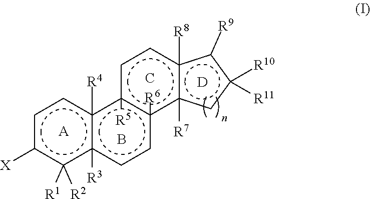

wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

49. The composition for use according to embodiment 46 or 47, wherein the one or more compounds of formula (II) is cholesterol and/or one or more phytosterols.

50. The composition for use according to any one of embodiments 46, 47 or 49, wherein the composition comprises two or more compounds of formula (II).

51. The composition for use according to embodiment 50, wherein one of the two or more compounds of formula (II) is cholesterol.

52. The composition for use according to embodiment 50 or 51, wherein one or more compounds of the two or more compounds of formula (II) is one or more phytosterols.

53. The composition for use according to embodiment 48, wherein the one or more compounds of formula (I) is cholesterol and/or one or more phytosterols.

54. The composition for use according to embodiment 48 or 53, wherein the composition comprises two or more compounds of formula (I).

55. The composition for use according to embodiment 54, wherein one of the two or more compounds of formula (I) is cholesterol.

56. The composition for use according to embodiment 54 or 55, wherein one or more compounds of the two or more compounds of formula (I) is one or more phytosterols.

57. The composition for use according to any one of embodiments 46 to 56, wherein the subject is suffering from an infectious disease, optionally wherein the infection is a bacterial infection, a viral infection or a parasitic infection.

58. The composition for use according to any one of embodiments 46 to 57, wherein the disease is sepsis.

59. The composition for use according to any one of embodiments 46 to 56, wherein the subject is suffering from a non-infectious disease, optionally wherein the disease is ischaemic heart disease or drug toxicity.

60. The composition for use according to any one of embodiments 46 to 59, wherein the composition is administered intravenously to the subject.

61. The composition for use according to any one of embodiments 46 to 60, wherein the composition further comprises a pharmaceutically acceptable carrier.

62. The composition for use according to embodiment 61, wherein the carrier is a nanocarrier.

63. The composition for use according to embodiment 61 or 62, wherein the carrier is a liposome, micelle, nanoparticle, nanoworm or nanorod.

64. The composition for use according to any one of embodiments 46 or 47, 49 to 52, or 57 to 63, wherein the composition comprises cholesterol and one or more other compounds of formula (II), and the concentration ratio of the one or more other compounds of formula (II) to cholesterol is from 75:25 to 25:75.

65. The composition for use according to any one of embodiments 46 or 47, 49 to 52, or 57 to 64, wherein:
(a) the composition comprises cholesterol and one or more other compounds of formula (II);
(b) the composition further comprises a pharmaceutically acceptable carrier; and
(c) the pharmaceutically acceptable carrier comprises cholesterol, and
the concentration ratio of the one or more other compounds of formula (II) in the composition to cholesterol in the composition and the pharmaceutically acceptable carrier is from 75:25 to 25:75.

66. The composition for use according to any one of embodiments 46 or 47, 49 to 52, or 57 to 64, wherein:
(a) the composition comprises one or more compounds of formula (II) but not cholesterol;
(b) the composition further comprises a pharmaceutically acceptable carrier; and
(c) the pharmaceutically acceptable carrier comprises cholesterol, and
the concentration ratio of the one or more compounds of formula (II) that is not cholesterol in the composition, to cholesterol in the pharmaceutically acceptable carrier, is from 75:25 to 25:75.

67. The composition for use according to any one of embodiments 64 to 66, wherein the concentration ratio of the one or more other compounds of formula (II) to cholesterol, the concentration ratio of the one or more other compounds of formula (II) to cholesterol in the composition and the carrier, or the concentration ratio of the one or more compounds of formula (II) that is not cholesterol in the composition to cholesterol in the carrier is about 50:50.

68. The composition for use according to any one of embodiments 46 or 47, 49 to 52, or 57 to 67, wherein when at least one of the one or more compounds with formula (II) or two or more compounds of formula (II) is a phytosterol, the phytosterol is selected from the group consisting of: sitosterol, campesterol, stigmasterol, campestanol, brassicasterol, ergosterol, lupeol, cycloartenol, and sitostanol.

69. The composition for use according to any one of embodiments 48, or 53 to 63, wherein the composition comprises cholesterol and one or more other compounds of formula (I), and the concentration ratio of the one or more other compounds of formula (I) to cholesterol is from 75:25 to 25:75.

70. The composition for use according to any one of embodiments 48, or 53 to 63, or 69, wherein:
(a) the composition comprises cholesterol and one or more other compounds of formula (I);
(b) the composition further comprises a pharmaceutically acceptable carrier; and
(c) the pharmaceutically acceptable carrier comprises cholesterol, and
the concentration ratio of the one or more other compounds of formula (I) in the composition to cholesterol in the composition and the pharmaceutically acceptable carrier is from 75:25 to 25:75.

71. The composition for use according to any one of embodiments 48, 53 to 63, or 69 to 70, wherein:
(a) the composition comprises one or more compounds of formula (I) but not cholesterol;
(b) the composition further comprises a pharmaceutically acceptable carrier; and
(c) the pharmaceutically acceptable carrier comprises cholesterol, and
the concentration ratio of the one or more compounds of formula (I) that is not cholesterol in the composition, to cholesterol in the pharmaceutically acceptable carrier, is from 75:25 to 25:75.

72. The composition for use according to any one of embodiments 69 to 71, wherein the concentration ratio of the one or more other compounds of formula (I) to cholesterol, the concentration ratio of the one or more other compounds of formula (I) to cholesterol in the composition and the carrier, or the concentration ratio of the one or more compounds of formula (I) that is not cholesterol in the composition to cholesterol in the carrier is about 50:50.

73. The composition for use according to any one of embodiments 48, 53 to 63, or 69 to 72, wherein when at least one of the one or more compounds with formula (I) or two or more compounds of formula (I) is a phytosterol, the phytosterol is selected from the group consisting of: sitosterol, campesterol, stigmasterol, campestanol, brassicasterol, ergosterol, lupeol, cycloartenol, and sitostanol.

74. The method of claims 1 to 28, or the composition for use of claims 46 to 73, wherein the subject is a human.

75. The method of any one of claims 18 to 28, the composition of any one of claims 35 to 45, or the composition for use of any one of claims 63 to 73, wherein when the carrier is a liposome, the one or more compounds with formula (I) are formulated within the liposome.

1. A method for the treatment of a disease associated with myocardial depression comprising administering to a subject in need thereof a therapeutically effective amount of a composition comprising one or more compounds of formula (II) or a pharmaceutically acceptable salt, hydrate, prodrug or stereoisomer thereof, 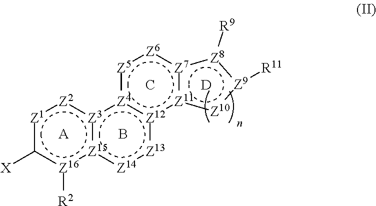
wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹ is selected from hydrogen and optionally substituted C₁₋₂₀ alkyl, or may be absent; and
n is 0, 1 or 2;
provided that no more than four of Z¹ to Z¹⁶ are NH or N.

2. A method of increasing adrenergic signalling responsiveness in a subject in need thereof, comprising administering to the cell, tissue or subject a therapeutically effective amount of a composition comprising one or more compounds of formula (II) or a pharmaceutically acceptable salt, hydrate, prodrug or stereoisomer thereof, 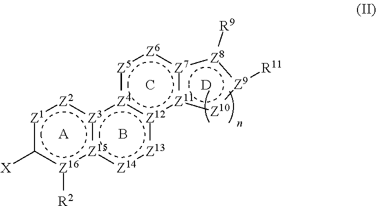
wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

3. The method according to claim 1, wherein the one or more compounds of formula (II) is cholesterol and/or one or more phytosterols, optionally wherein said one or more phytosterols are selected from the group consisting of: sitosterol, campesterol, stigmasterol, campestanol, brassicasterol, ergosterol, lupeol, cycloartenol, and sitostanol.

4. (canceled)

5. The method according to claim 1, wherein the one or more compounds of formula (II) are one or more compounds of formula (I), 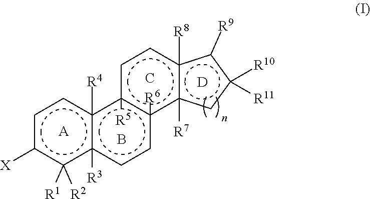
wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

6.-7. (canceled)

8. The method according to claim 1 wherein the subject is suffering from a disease caused by an infection, optionally wherein the infection is a bacterial infection, a viral infection or a parasitic infection, further optionally wherein the disease is sepsis.

9. (canceled)

10. The method according to claim 1 wherein the composition further comprises a pharmaceutically acceptable carrier, optionally wherein the carrier is a liposome, micelle, nanoparticle, nanoworm or nanorod.

11. (canceled)

12. A pharmaceutical composition comprising: (i) cholesterol and/or (ii) one or more other compounds with formula (II), or a pharmaceutically acceptable salt, hydrate, prodrug or stereoisomer thereof, 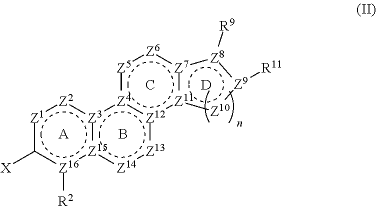
wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₂₀ alkyl;
Z¹ is selected from CH₂, CF₂, CH, CF, NH and N;
Z² is selected from CH₂, CF₂, CH, CF, NH and N;
Z³ is selected from CR⁴ and N;
Z⁴ is selected from CR⁵ and N;
Z⁵ is selected from CH₂, CF₂, CH, CF, NH and N;
Z⁶ is selected from CH₂, CF₂, CH, CF, NH and N;
Z⁷ is selected from CR⁸ and N;
Z⁸ is selected from CH, CF and N;
Z⁹ is selected from CR¹⁰ and N;
each Z¹⁰ is independently selected from CH₂, CF₂, CH, CF, NH and N;
Z¹¹ is selected from CR⁷ and N;
Z¹² is selected from CR⁶ and N;
Z¹³ is selected from CH₂, CF₂, CH, CF, NH and N;
Z¹⁴ is selected from CH₂, CF₂, CH, CF, NH and N;
Z¹⁵ is selected from CR³ and N;
Z¹⁶ is selected from CR¹ and N;
R¹ is selected from hydrogen, fluorine and optionally substituted C₁₋₂₀ alkyl;
R² is selected from hydrogen and optionally substituted C₁₋₂₀ alkyl, or may be absent;
R³ is selected from hydrogen, fluorine and optionally substituted C₁₋₂₀ alkyl, or may be absent;
R⁴ is selected from hydrogen, fluorine and optionally substituted C₁₋₂₀ alkyl, or may be absent, and R⁵ is selected from hydrogen, fluorine and optionally substituted C₁₋₂₀ alkyl, or may be absent, or R⁴ and R⁵ are together C₁ alkylene optionally substituted with at least one C₁₋₂₀ alkyl, such that R⁴ and R⁵ together with Z³ and Z⁴ form a three-membered ring;
R⁶ is selected from hydrogen, fluorine and optionally substituted C₁₋₂₀ alkyl, or may be absent;
R⁷ is selected from hydrogen, fluorine and optionally substituted C₁₋₂₀ alkyl, or may be absent;
R⁸ is selected from hydrogen, fluorine and optionally substituted C₁₋₂₀ alkyl, or may be absent;
R⁹ is selected from optionally substituted C₁₋₂₀ alkyl, C₂₋₂₀ alkenyl and C₂₋₂₀ alkynyl and R¹⁰ is selected from hydrogen and optionally substituted C₁₋₂₀ alkyl, or R⁹ and R¹⁰ are together C₃-C₅ alkylene optionally substituted with at least one C₁₋₂₀ alkyl, C₂₋₂₀ alkenyl or C₂₋₂₀ alkynyl, such that R⁹ and R¹⁰ together with Z⁸ and Z⁹ form a 5-7 membered ring;
R¹¹ is selected from hydrogen and optionally substituted C₁₋₂₀ alkyl, or may be absent; and
n is 0, 1 or 2;
provided that no more than four of Z¹ to Z¹⁶ are NH or N.

13. The composition of claim 12, wherein the one or more compounds of formula (II) are one or more compounds of formula (I), 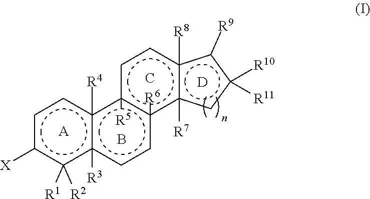
wherein:
each of the rings A, B, C and D independently contain from 0 to 3 double bonds;
X is selected from OH, NH₂, NHR, CO₂H, CO₂R, SO₂H and SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

14. The composition according to claim 13, wherein at least one of the one or more other compounds with formula (I) is a phytosterol, optionally wherein said one or more phytosterols are selected from the group consisting of: sitosterol, campesterol, stigmasterol, campestanol, brassicasterol, ergosterol, lupeol, cycloartenol, and sitostanol.

15. The composition according to claim 13, wherein the composition comprises cholesterol and one or more other compounds of formula (I), and the concentration ratio of the one or more other compounds of formula (I) to cholesterol is (i) from 75:25 to 25:75 or (ii) about 50:50.

16. The composition according to claim 13, wherein:
(a) the composition comprises cholesterol and one or more other compounds of formula (I);
(b) the composition further comprises a pharmaceutically acceptable carrier; and
(c) the pharmaceutically acceptable carrier comprises cholesterol, and
the concentration ratio of the one or more other compounds of formula (I) in the composition to cholesterol in the composition and the pharmaceutically acceptable carrier is (i) from 75:25 to 25:75 or (ii) about 50:50.

17. The composition according to claim 13, wherein:
(a) the composition comprises one or more compounds of formula (I) but not cholesterol;
(b) the composition further comprises a pharmaceutically acceptable carrier; and
(c) the pharmaceutically acceptable carrier comprises cholesterol, and
the concentration ratio of the one or more compounds of formula (I) that is not cholesterol in the composition, to cholesterol in the pharmaceutically acceptable carrier, is (i) from 75:25 to 25:75 or (ii) about 50:50.

18. (canceled)

19. The composition according to claim 12, wherein the composition further comprises a pharmaceutically acceptable carrier, optionally wherein the carrier is a liposome, micelle, nanoparticle, nanoworm or nanorod.

20.-31. (canceled)

32. The method of claim 1 wherein the subject is a human.

33. The method of claim 10, wherein when the carrier is a liposome, the one or more compounds with formula (II) or (I) are formulated within the liposome.

34. The method according to claim 2, wherein the one or more compounds of formula (II) is cholesterol and/or one or more phytosterols, optionally wherein said one or more phytosterols are selected from the group consisting of: sitosterol, campesterol, stigmasterol, campestanol, brassicasterol, ergosterol, lupeol, cycloartenol, and sitostanol.

35. The method according to claim 2, wherein the one or more compounds of formula (II) are one or more compounds of formula (I), 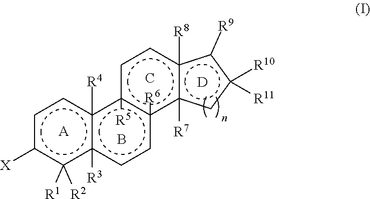
wherein:
each of the rings A, B, C, and D independently contain from 0 to 3 double bonds;
X is selected from OH, NH₂, NHR, CO₂H, CO₂R, SO₂H, SO₂R, wherein each R is independently C₁₋₆ alkyl;
R¹ is selected from hydrogen and C₁₋₆ alkyl;
R² is selected from hydrogen and C₁₋₆ alkyl, or may be absent;
R³ is selected from hydrogen and C₁₋₆ alkyl, or may be absent;
R⁴ is selected from hydrogen and C₁₋₆ alkyl, or may be absent, and R⁵ is selected from hydrogen and C₁₋₆ alkyl, or may be absent, or R⁴ and R⁵ together with the adjacent carbons in ring B form cyclopropane optionally substituted on the carbon which does not form part of ring B with at least one C₁₋₆ alkyl;
R⁶ is selected from hydrogen and C₁₋₆ alkyl, or may be absent;
R⁷ is selected from hydrogen and C₁₋₆ alkyl, or may be absent;
R⁸ is selected from hydrogen and C₁₋₆ alkyl, or may be absent;
R⁹ is selected from C₁₋₁₂ alkyl, C₂₋₁₂ alkenyl and C₂₋₁₂ alkynyl and R¹⁰ is selected from hydrogen and C₁₋₆ alkyl, or R⁹ and R¹⁰ together with the adjacent carbons in ring D form a C₅₋₇ cycloalkane ring wherein the carbon atoms that do not also form part of ring D may be optionally substituted with at least one C₁₋₆ alkyl, C₂₋₆ alkenyl or C₂₋₆ alkynyl;
R¹¹ is selected from hydrogen and C₁₋₆ alkyl, or may be absent; and
n is 0, 1 or 2.

36. The method according to claim 2, wherein the subject is suffering from a disease caused by an infection, optionally wherein the infection is a bacterial infection, a viral infection or a parasitic infection, further optionally wherein the disease is sepsis.

37. The method according to claim 2, wherein the composition further comprises a pharmaceutically acceptable carrier, optionally wherein the carrier is a liposome, micelle, nanoparticle, nanoworm or nanorod.

38. The method according to claim 2, wherein the subject is a human.
